(19) United States
(12) Patent Application Publication
(10) Pub. No.: US 2024/0367165 A1
(43) Pub. Date: Nov. 7, 2024

(54) SAFE SELF-TESTING OF MULTIPLEX BIOMARKERS IN BIOFLUIDS

(71) Applicant: ARIZONA BOARD OF REGENTS ON BEHALF OF THE UNIVERSITY OF ARIZONA, Tucson, AZ (US)

(72) Inventors: Frederic ZENHAUSERN, Phoenix, AZ (US); Ali FATTAHI, Phoenix, AZ (US); Jerome LACOMBE, Phoenix, AZ (US)

(21) Appl. No.: 18/683,189

(22) PCT Filed: Aug. 12, 2022
(86) PCT No.: PCT/US22/40241

§ 371 Date: Feb. 12, 2024

Related U.S. Application Data
(60) Provisional application No. 63/233,058, filed on Aug. 13, 2021.

(51) Int. Cl. B01L 3/00

(52) U.S. Cl. CPC B01L 3/5023; B01L 2200/025; B01L 2200/10; B01L 2300/0654; B01L 2300/0681; B01L 2300/069; B01L 2300/1805

(57) ABSTRACT

Provided herein are devices for self-testing for a plurality of biomarkers from a liquid biofluid sample. and related methods of using the devices to test for the biomarkers presence or absence. The devices comprise a specially configured vertical flow biosensor to provide a device platform that is safe and can be used at-home/in the field without need for any specialized equipment or external power sources.

### CROSS-REFERENCE TO RELATED APPLICATIONS

This application claims the benefit of priority of U.S. Provisional Patent App. No. 63/233,058, filed Aug. 13, 2021, which is hereby incorporated by reference in its entirety.

### STATEMENT REGARDING FEDERALLY SPONSORED RESEARCH OR DEVELOPMENT

This invention made with Government support under Grant Numbers NNX16A069A awarded by the National Aeronautics and Space Administration (NASA) and 5U01AI148307-02 awarded by the National Institutes of Health. The Government has certain rights in this invention.

### BACKGROUND

Provided herein are devices that are useful for safe self-testing of multiplex biomarker panels in biofluids, including without powered instruments and that can be performed at home or while mobile.

Point-of-care (POC) assays are essential diagnostic platforms that allow fast health monitoring and disease testing with minimal equipment. Therefore, there is a critical need to develop autonomous platforms providing a detailed medical diagnosis to help individuals make a decision on personal care or self-therapeutic actions. With the sudden COVID-19 pandemic, we developed a platform which can be suitable for an untrained user to self-collect a specimen of biofluid (e.g. blood, saliva or urine) and process it safely without being exposed to hazardous chemicals or reagents (see, e.g., US 2022/0001378 “Smart Storage Container for Health Logistics”; US 2021/0199651 “Vertical Flow Molecular Assay Apparatus”; US 2021/0030347 “Integrated Device for Self-Collecting and Automated Pre-Processing of Biological Fluids”; WO 2022/026831 “Safe Specimen Transportation Isolation Container”; WO 2022/072876 “Biofluid Self-Collection and Processing Device”).

The devices and methods provided herein address the need in the art by providing a safe and easy to use collection and, more specifically, a testing system that can, without external power sources or interventions, provide an optically-detectable indication related to presence or absence of biomarkers in a biofluid sample.

### SUMMARY

Provided herein is an apparatus platform for detecting biomarkers from a liquid biofluid sample which does not require any power supply or any specialized readout instrumentation, and that can be performed without the need of medical facilities, in less than a processing time, such as a processing time that is less than 1 hour, from sample collection to data analysis The apparatus is also compatible for running under a variety of environmental conditions including against gravity or at zero g space application. Typically, a user will collect a small volume of whole blood from a finger prick and plasma will be isolated using a point-of-care (POC) microfluidic cartridge for preparing proteins to be detected quickly with the device before visualizing by eye or imaging the results with a software installed onto a smart phone. A direct collection of saliva is also amenable for this apparatus. As such, the devices provided herein are particularly suited for evaluation of the presence or absence of a biomarker.

The biomarker can be indicative of a biological state, reflecting health or wellbeing of an individual. Applications include, but are not limited to, assessing infection or contamination, radiation poisoning and/or presence of an infectious agent. For example, the biomarker can relate to a pandemic. As but one example, with the emergence of COVID-19 pandemic, the device platform can be adapted to monitor SARS-COV-2 infection using both N gene amplification for virus detection and the capture of anti-S protein Ab to identify immune response against SARS-Cov-2. Because the devices and methods provided herein are compatible with any of a range of biomarkers from any of a range of biological applications, the devices and methods are characterized as a platform for detecting biomarkers.

Results indicate hat on the same membrane the device can detect as low as 100 copies of SARS-Cov-2 and about 12 ng of anti-S protein Ab. The device is validated for the detection of C-reactive protein (CRP), albumin, IgM, IgG and IgA and also for other protein markers and/or genes related to biodosimetry. The devices utilize fluidics, including through a combination of tubes and caps that can allow the sample, mixed with assay buffer, to cross the membrane by the action of an adsorptive nano-pad located underneath the membrane which drives fluid by capillary forces. Experiments indicate that the devices can run independently of its orientation, i.e. with the pad capillary motion working even against gravity, or possibly at zero gravity.

Also provided herein are methods of collecting biofluid samples and having a user run the assay, including for a method of bioanalytical detection of a biological parameter, without any outside assistance by using any of the devices provided herein.

Provided herein is a device for self-testing for a plurality of biomarkers comprising: a sample inlet configured to receive a liquid biofluid sample; a sample pre-processing module fluidically connected to the sample inlet to provide a pre-processed biofluid sample; a filter in fluidic contact with the liquid biofluid sample or the pre-processing module to provide a filtered fluid sample; a powerless heat source in thermal contact with the pre-processed biofluid sample and/or the filtered fluid sample for controlled temperature of the pre-processed biofluid sample and/or filtered fluid sample; a vertical flow biosensor (VFB) comprising a multiplex membrane in fluidic contact with the pre-processed biofluid sample for multiplex detection of the plurality biomarkers in the liquid biofluid sample.

The devices can be described as self-powered. This refers that no external power source, such as an electrical power source, is required to run the assay implemented by the device.

The liquid biofluid sample is a saliva sample, a plasma sample, a blood sample, a urine sample, a sputum sample, a semen sample, a vaginal discharge sample, a tear fluid, a breath condensation droplet, a CSF fluid biopsy, plural effusion, or other biological effusion. Preferably, the liquid biofluid sample is a saliva sample.

The sample pre-processing module may comprises one or more of: a filter in fluidic contact with the liquid biofluid sample to provide a filtered biofluid sample; a mucus removal reagent (MRR) fluidically connected to the sample inlet to introduce the MRR to the liquid biofluid sample that is a saliva sample, wherein MRR removes mucus from the saliva sample and reduces a viscosity of the saliva sample; or a rheological property adjuster, such as chitosan, mucoadhesive biopolymers, or polymers with electrostatic charges or conformation with hydrophobic and hydrophilic domains controlling molecular and environmental interactions to adjust a rheological parameter of the liquid biofluid sample. The sample pre-processing module may also facilitate adjustment of the liquid biofluid sample pH by a pH adjuster, concentration, or other component that modulates interaction forces (e.g., hydrophobicity) between the biofluid sample and the environment. The filter may correspond to the filter in fluidic contact with the “upstream” liquid biofluid sample, or it may be an additional filter that provides further filtering to a “pre-filtered” liquid sample.

The plurality of biomarkers may comprise one or more of: one or more markers of an infectious agent (e.g., gene or protein from a bacteria, virus, fungi, parasites) and fragments thereof (polypeptides, polynucleotides); one or more markers of a host immune response (e.g., antibodies, T-cells); one or more vaccine markers; one or more cancer biomarkers; one or more nutrition or metabolic biomarkers; one or more auto-immune disorder biomarkers; one or more cardiovascular biomarkers; one or more genetic disorder biomarkers; or one or more environmental biomarkers.

The device may have a powerless heat source comprising: a chemical heat source comprising reagents for an exothermic chemical reaction to provide a biological sample temperature range of between 34° C. and 95° C. to activate at least one step of an amplification reaction of a biological component in the liquid biofluid sample. The amplification reaction may be by PCR; LCR; isothermal; and/or RCA.

The MRR may comprise a polymeric-based solution configured to interact with mucin in the liquid biological sample that comprises saliva. The MRR may be provided to the sample pre-processing module, such as a saliva collection device where a volume of saliva is contained. The device may further comprise: a filter substrate material having an average pore size selected to remove debris and food residue from the saliva; a substrate material having a physical parameter (e.g. low density) and chemical property (e.g., inert) configured to establish an interface with the biofluid sample for directing one or more analytes in the biofluid sample to the VFB.

The VFB is an electromagnetic power-free and is fluidically activated by intra-molecular (e.g., capillary) or external forces (e.g., gravity), comprising: a membrane housing; a multiplex membrane and a sample absorbent pad in fluidic contact with the membrane, wherein the membrane and the sample absorbent pad are positioned in the membrane housing and the absorbent pad is fluidically connected to the liquid biological sample, wherein biomarkers from the biological liquid sample are provided to the multiplex membrane. The provided to aspect can be without an external power-source, such as a pump, including by capillary action and/or gravity. Of course, the VFB can be adapted to receive fluid flow of biomarkers under an external energy source, such as a battery-powered syringe pump.

The VFB may be in a multi-layer configuration, such as in a stacked-pad configuration, including contained in a membrane housing. The stacked-pad may include one or more of: a buffer pad configured to store assay buffer; a sample pad; the multiplex membrane, such as a polyethersulfone membrane; a conjugation pad; a retarding pad; and a flow directing pad.

The device may further comprise an imager for optical detection of presence or absence of the plurality of biomarkers in the VFB, wherein the imager is optionally a magnifying lens and/or a portable reader (including a hand-held camera or a smart-phone imaging camera). The imager may comprise a magnifying lens configured to optically align with at least one lens of a smart phone or a commercially-available ancillary configured to perform biomarker analysis. A commercially-available ancillary may include a camera, a sensor that grabs images, or other optical detector operably connected to a controller in the form of software and/or hardware to analyze the image and identify presence or absence of an optical signal that indicates the presence or absence of a biomarker.

The device may further comprise a point-of-care microfluidic cartridge for preparing proteins in the liquid biological sample for detection by the VFB.

The devices and methods provided herein are compatible with a range of biomarkers. Examples of particularly relevant biomarkers include one or more of: human antibodies to an infectious agent, such as human anti-SARS-COV2 antibodies; total IgM, IgC, IgA, or combinations thereof; inflammatory or stress response protein(s), such as CRP; SARS-COV-2 N-gene; Human CDKN1A, DDB2 and MRPS5 gene; and/or a small molecule (e.g., a toxin) or other biomolecular species indicative of a disease condition or an environmental exposure (e.g., radiation, extreme climate condition).

The device biomarker detection may be independent of device orientation and operable under zero-g conditions.

In a particularly useful configuration, the multiplex membrane comprises biomarker detectors to detect: presence or absence of a virus; and presence or absence of a host immune response. In this manner, the multiplex character can provide information about both presence or absence of a virus and the immunization status of an individual, including as reflected by presence or absence of antibodies. The immune response may be to confirm efficacy of a vaccine and the potential severity of a viral infection with attendant outcome risks.

Also provided herein are methods of using any of the disclosed devices. For example, the method may be a method of bioanalytical detection of a biological parameter. The method may comprise the steps of providing any of the devices described herein and introducing the liquid biofluid sample to the sample inlet. Debris in the liquid biological sample is removed by the pre-processing module, including by a filter in the pre-processing module. The pre-processed filtered liquid biofluid sample mixture is introduced to the multiplex membrane. This introduction is preferably without any external power, such as by capillary force or by the force of gravity. Any relevant biomarkers in the liquid biofluid sample may interact with the multiplex membrane and be optically detected. In this manner, detection of a biological parameter is obtained. For example, the method further comprises optically detecting the one or more biomarkers that have interacted with the multiplex membrane, to thereby obtain a diagnostic parameter. The optical detection may be by eye or may be with a camera system and ancillary analysis system to determine presence/absence of a biomarker. As the method may be a multiplex detection of a plurality of biomarkers, there may be a spatial pattern of biomarker detection agents (e.g., polynucleotides, polypeptides) and controls for automated read-out of presence/absence of various biomarkers. With an understanding of the specific biomarkers that are present/absent, a biological parameter is determined. A straightforward biological parameter relates to infection status, with a biomarker that is a viral biomarker (protein, polynucleotide sequence) that is detected indicative of a biological parameter that is “positive infection” or that is not detected a biological parameter that is a “negative infection.”

The removing step may comprise mixing the liquid biofluid sample with a MRR and filtering the mixed liquid biofluid sample and MRR to provide a filtered liquid biological sample.

An important aspect of the devices and related methods herein is that they can be performed on-demand and at-home by individuals who have not special training. Furthermore, the methods are also characterized as being fast methods for obtaining a biological parameter, such as a total method time of less than one hour, less than 30 minutes, or less than 15 minutes. This fast time arises because no special instruments are required, beyond at most an imagining system, such as a smart phone having a camera and attendant application software for analyzing optical output from the multiplex membrane that has interacted with a liquid sample.

Examples of biological parameters include one or more of: determination of a past infection event; current infection status; immunity status; donor compatibility; vaccine quality control; radiation biodosimetry; prediction: prediction of treatment efficacy; risk assessment: assessment of disease susceptibility; screening/detection: indication of the presence of the disease (early detection); prognosis: assessment of disease aggressiveness; monitor: monitoring of disease recurrence and therapeutic response; and/or pharmacological response (e.g. drug efficacy, dose response, safety, genotype . . . ); screening/detection: indication of the presence of the disease (early detection); and/or prognosis: assessment of disease aggressiveness; monitor: monitoring of disease recurrence and therapeutic response.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 summarizes a sample preparation process, including a quick sample preparation using mucus removal agent (MRR) and formation of the Floating Gel. The aqueous solution is run directly into the Vertical Flow Biosensor (VFB) tends to generate a strong background. Addition of a filtering step, such as with a 0.45 μm low protein absorption polyether sulfone membrane, reduces background and provides a cleaner signal (right panel).

FIGS. 2A-2D summarizes different geometries and functional characteristic for heat boxes of the device. FIG. 2A are photographs of a power-free ISOCOV Heat Box to maintain a reaction temperature, such as in a range of 38° C.-42° C. FIG. 2B is a temperature profile of two jackets with different wall thickness demonstrating that a different temperature range can be selected by changing the jacket thickness and/or jacket material composition (25 mm thickness results in 47° C.; 35 mm thickness results in 43° C.). FIG. 2C is a comparison of benchtop system with ISOCOV Heat Box. FIG. 2D is the ISOCOV signal at different copy numbers of N gene (0-100 K). The limit of detection (LoD) of ISOCOV is about 10 copies/μL.

FIGS. 3A-3D illustrate a device and related steps for using the device. FIG. 3A shows different parts of the VFB and brief workflow summary of the assay. FIG. 3B illustrates a safe and effective locking mechanism for holding the membrane on running position which is in contact with absorption pad and moving to dry mode by repositioning to position 2 to detach the membrane from absorption pad. FIG. 3C shows removing conjugation pad holder from membrane housing and wet pad after running the assay without touching by hand. FIG. 3D shows the presence of indicator on absorption pad, thereby helping a user to monitor the endpoint of test.

FIG. 4A shows the effort of sponge material on signal intensity against gravity assay using c-reactive protein (CRP) as a biomarker. FIG. 4B shows the pad holder and its pad layers, which layers are stacked to the holder and each other by double-sided tape.

FIG. 5A illustrates the effect of plasma volume on signal intensity and FIG. 5B illustrates the effect of assay time on signal intensity, for a CRP detection assay. FIG. 5C is a stability study of immobilized CRP Ab-GNP on conjugation pad over the time period of 1 day, 3 days and 1 week. FIG. 5D is a Limit of Detection (LoD) determination for S-protein Ab. LoD was 12.5 ng/reaction. FIG. 5E summarizes 1SARS-COV-2 antibody test using human saliva and plasma as specimens.

FIG. 6A is a multiplex assay for simultaneous detection of N-gene and SARS-COV-2 antibody. FIG. 6B is a quantitative analysis of signal intensity in multiplex assay of N-gene and SARS-COV-2 antibody.

FIG. 7 is a photograph of a device wherein various tube and caps contain various components for optical detection of biomarkers.

FIG. 8 illustrates membrane layout (top-left) for testing of various nucleic acid biomarkers (CDKN1A and DDB2) with controls and a housekeeping gene (MARPS5).

FIG. 9A is a plot of gene expression detected by qPCR as a function of radiation dose. FIG. 9B is a plot of gene expression detected by a device of the instant invention (e.g., device having a vertical flow biosensor) as a function of radiation dose. Both techniques detect a dose-response curve.

FIGS. 10A-10B illustrate multiplex detection of biomarkers by a vertical flow biosensor by simultaneous detection of both gene (SARS-COV-2 N gene for viral particle detection) and protein (human anti-S protein IgG to assess immune response to SARS-COV-2) on the same membrane. FIG. 10A illustrates the biomarker detection layout on the membrane of a vertical flow biosensor containing device. FIG. 10B illustrates the results for various samples containing high, low or no of N gene (viral) and anti-S protein antibody.

FIGS. 11A-11C: FAST-DOSE biomarker radiosensitivity. Human blood samples are irradiated ex vivo with X-rays (0 to 5 Gy) and biomarker expression is measured in gated lymphocyte populations on Days 1 and 2 post x-irradiation using the % positive metric. Left panel: Fold changes for the top biomarkers are calculated as 3Gy/0Gy (n=total of 20 combined across biomarkers tested). Data are expressed as mean+SEM. Dose response curves based on fold the fold change in % positive biomarker expression for (middle panel) BAX and (right panel) phospho-p53 (Ser 37). R² values represent correlation coefficient of linear regression and p values represent significance of linear regression.

FIG. 12 illustrates ease of deployment of the device and kit to the field, with no power sources or specialized equipment required to detect genes and proteins.

FIG. 13A summarizes the principle of the multiplex sandwich immunoassay performed (e.g., VFB of the device is a VFI). FIG. 13B shows an actual membrane with positive signal after a VFI run.

FIG. 14 is a table summary of some multiplex assays illustrating the improved limit of detection (LOD) for three different Tier 1 biothreats.

FIG. 15 illustrates the optical readout to detect one or more biomarkers can be implemented with a smart handheld device such as an iPhone with an App. The App performs image analysis and result reporting.

FIG. 16 is an overall workflow process for multiplex detection of various biomarkers from a saliva sample.

FIG. 17 Limit of detection from saliva samples spiked with N gene (left panel) and anti-SARS-COV-2 Spike(S) glycoprotein IgG (right panel).

FIG. 18 Use of the VFB with a saliva collector.

FIG. 19 Competitive assay on VFI to detect saxitoxin. Similar configurations are used for nAbs.

FIG. 20 Comparison of the standard column-based PCR purification protocol (left panel) with the integrated magnetic bead purification (right panel).

FIG. 21A-21B Images of optical output from control plasma from non-infected, non-vaccinated donor (FIG. 21A) and plasma from vaccinated donor (FIG. 21B).

FIG. 22 is a representative device schematic.

FIG. 23 Flow-chart summary of a method of detecting a biological parameter.

### DETAILED DESCRIPTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In general, the terms and phrases used herein have their art-recognized meaning, which can be found by reference to standard texts, journal references and contexts known to those skilled in the art. The following definitions are provided to clarify their specific use in the context of the invention.

“Self-testing” refers to a device that can be operated and read-out by the user, and does not require any specialized personal, equipment or laboratory procedures.

“Biomarkers” is used broadly herein to refer to an analyte of interest in the biofluid sample. The biomarker, depending on the application of interest, can range from a protein, a gene, a polypeptide sequence, a polynucleotide sequence, an antibody, a cell type (e.g., cancer cell, T-cell). The devices and methods provided herein are compatible with a range of biomarkers, including any molecule that can be bound to an antibody, such as an antibody placed on a multiplex membrane that is part of the vertical flow biosensor. “Multiplexed” refers to the device being able to detect more than one biomarker, including biomarkers from a panel of related biomarkers where the plurality of biomarkers can provide additional information and/or increase device reliability.

“Sample pre-processing module” refers to components in the device that are capable of processing the biofluid sample to make the sample suitable for detection of biomarkers in the biofluid sample by the VFB. Accordingly, the specific structure of the sample pre-processing module depends on the application of interest, particularly the type of sample, and the biomarkers of interest. For example, for saliva a filter and/or MRR may be used to remove debris and unwanted biofluid constituents, such as mucin. Other structures include, but are not limited to, sorting filters, absorbents, reagents (e.g., enzymes, buffers, diluents, etc.). Representative function of the underlying structure includes the ability to control a rheological parameter, such as viscosity, so that liquid flow is achieved downstream to and in the VFB, and removal of potential interfering materials, whether that is large substances that could clog pores in the flow-through multiplex membrane or materials that can interfere with binding.

“Self-powered” or “powerless” refers to the devices and methods that do not require an external source of power, such as a battery or other power generation corresponding to an electromagnetic or electrochemical power source. Instead, the individual components themselves provide the necessary forces, such as a fluidic flow (e.g., by capillary flow, absorbent pads, etc.) or heat via a chemical reaction of reagents provided as part of the device. “Powerless” more particularly refers to a powerless heat source wherein the heat energy is generated without an active power source like a battery, but instead is by an exothermal reaction (e.g. phase change materials). “Self-powered” more particularly refers to a self-powered device that does not require an external electrical power source, but instead relies only on intrinsic physical forces (e.g. capillarity to move the fluid; exothermal chemical power for heat control, etc.) to run the assay.

Accordingly, a powerless heat source may be a chemical heat source wherein upon mixing of reagents, heat is generated. This is particularly suited for amplification reagents that occur at a defined temperature, without thermal cycling. Examples of amplification reactions include by PCR (polymerase chain reaction); LCR (ligase chain reaction); isothermal (LAMP or loop-mediated isothermal amplification); and/or RCA (rolling circle amplification).

“Operably connected” refers to the configuration of two components the connect, either directly or indirectly, but in a manner that maintains operability and functionality of each component. “Fluidically connected” refers to a configuration of two components for passage of a fluid such as a gas or a liquid, but in a manner that maintains operability and functionality of the individual components.

“Thermal contact” refers to two components that are positioned relative to each other such that heat can transfer between the components without adversely impacting the functionality of each component. The components may be described as proximate or adjacent to each other. Accordingly, there may or may not be intervening components between the components described as in thermal contact.

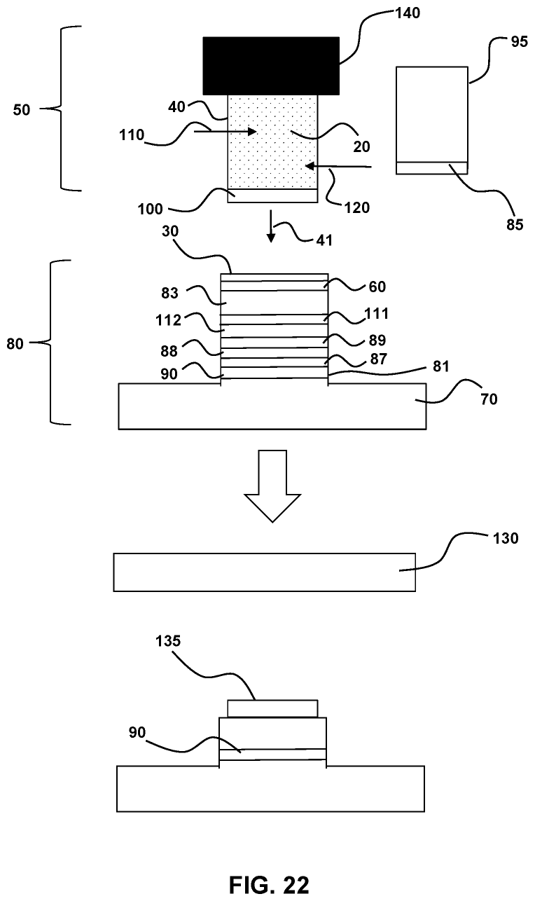

Referring to the figures, provided are devices and related methods for self-testing a plurality of biomarkers 20, including for detecting a biological parameter. With respect to FIG. 22, the vertical flow biosensor (VFB) 80 has a sample inlet 30 configured to receive a liquid biofluid sample 40, including from a sample pre-processing module 50 containing the liquid sample, such as saliva. The sample pre-processing module 50 and VFB 80 may reversibly connect to facilitate passage of pre-processed biofluid sample 41 into sample inlet 30 of the VFB 80. A filter 100, mucus removal reagent 110 and/or rheological property adjuster 120 can be used to in the preprocessing module 50 to provide a pre-processed biofluid sample 41 that is provided to the VFB 80. As desired, a point-of-care microfluidic cartridge 140 may be used to facilitate sample pre-processing, including with respect to protein purification Filter 60 may be positioned in the VFB 80. Powerless heat source 70, including a chemical heat source, can provide control of the fluid sample 40 41. The VFB may include a multiplex membrane 90. The resultant membrane with biomarker 91 interacting therewith can be imaged by imager 130.

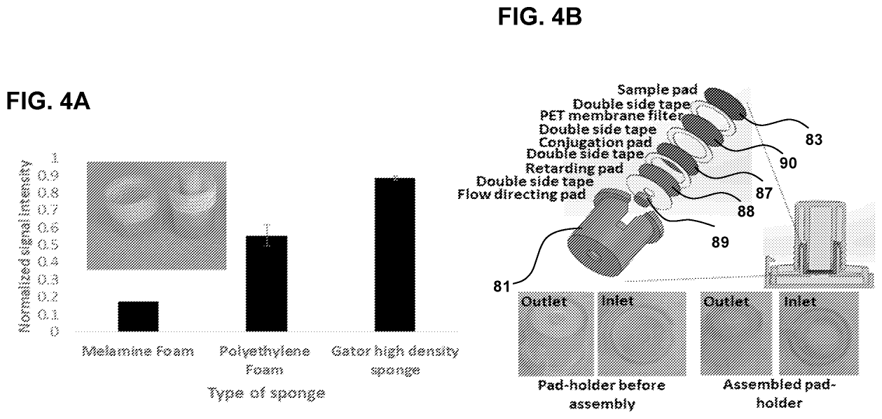

A reagent holder 95 may contain reagents necessary for the multiplex membrane to reliably interact with biomarkers 20, including reagents that can be delivered by buffer pad 85 that can be introduced to the sample and multiplex membrane by removably connecting to the membrane housing 81. The VFB may be in a stacked-pad configuration, including buffer pad 85, sample absorbent pad 83, multiplex membrane 90, conjugation pad 87, fluid flow retarding pad 88, and flow directed pad 89. See also FIG. 4B.

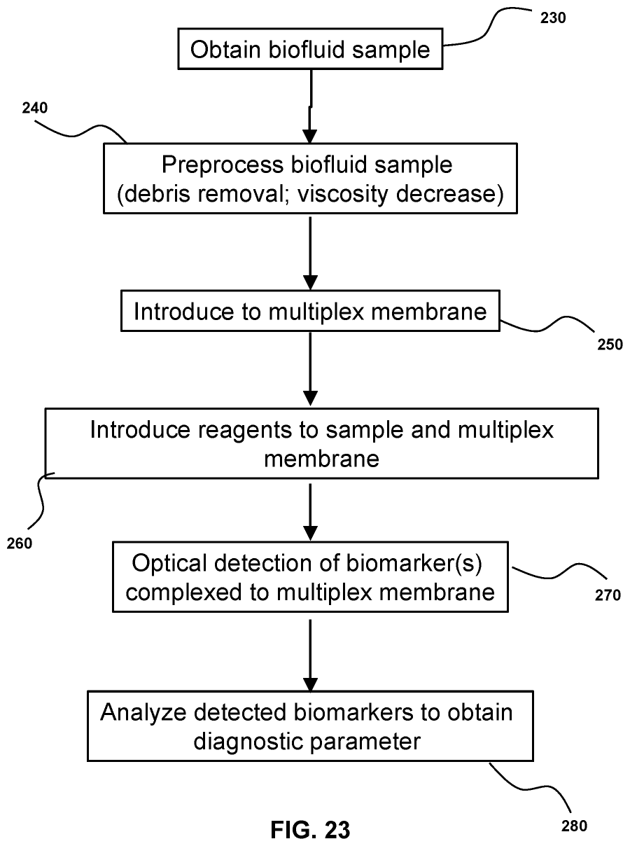

FIG. 23 summarizes a method of obtaining a diagnostic parameter from a biofluid sample. A biofluid sample is obtained 230 and preprocessed 240 so that the sample can be reliably introduced to the multiplex membrane 250. Reagents to facilitate interaction between the membrane and the biomarkers, more particularly capture agents positioned on the membrane, are introduced 260. Biomarker(s) complexed to the multiplex membrane are optically detected 270 and analyzed 280.

### Example 1: Device for at-Home Self-Testing

After more than one year of COVID tragedy, COVID vaccines open a new window to overcome the COVID19 crisis and return society to normalcy. Developing a new at-laboratory assay or diagnostic device for detecting covid provides a proof of concept for any of a range of infectious agents. The devices and methods provided herein facilitate at-home monitoring of infection and immunization and is the most efficient approach for helping to prevent, or at least manage, pandemic surges, including surges of COVID-19 disease as variants continue to evolve. By increasing the number of vaccinated populations, the main focus on “infection” is gradually shifting to the concern of vaccination's efficacy and duration of immunity. Differentiating between infectious agents, such as COVD-19 from flu and simple cold, is also of concern. Such differentiation can facilitate a return to normalcy by allowing those having a common cold to reliably confirm that another more dangerous infectious agent (e.g., COVID) is not a factor. At-home diagnostic devices, able to detect active infection and immunization level simultaneously, stand as the main option to address social concerns and psychologically support everyone to feel safe.

Currently, quantitative real-time reverse transcription-polymerase chain reaction (RT-qPCR) test is the golden standard molecular method for direct detection of a virus, while antibody test screens the body's response to the infection. The golden standard RT-qPCR is costly, slow, and labor-intensive. Recombinant Polymerase Amplification (RPA) and Loop-mediated Isothermal Amplification (LAMP) are POC alternatives for RT-qPCR, compatible with at-home applications. However, all the approved and underdeveloped POC-PCRs (e.g., All-IN-ONE, EIKON PCR, and Cue) are costly and incompatible for low resource environments and low-income countries. The SARS-COV-2 antibody and antigen test setting is more compatible with the POC test, and there is a higher chance of success for them as an at-home test. Although the FDA has granted Emergency Use Authorization (EUA) to 85 SARS-COV-2 antibody and other adaptive immune response tests as of July 12, 2021, none of those tests are approved for at-home application, and consumers need to pay $40-150 out of pocket that makes them relatively expensive.

Therefore, developing an at-home multiplex diagnostic kit to detect infection and screen the immunization is highly in demand; it can help prevent the spread of an infectious agent, such as SARS-COV-2, and determine vaccine effectiveness and immunity duration post-vaccination. The at-home test provided herein is user-friendly, low-cost, rapid, and safe and secure for the non-healthcare user.

Noninvasive sample collection and straightforward sample preparation are critical parameters to guarantee the safety of the non-healthcare user. To this point, saliva is the best specimen for an at-home test for COVID-19; the CDC approved saliva as a noninvasive specimen for the detection of SARS-COV-2 and SARS-COV-2 antibodies. Collecting a salivary sample is more convenient than nasal soap and sputum. However, the biomarkers' concentration in saliva is lower than that of other sources; and the high viscosity of saliva hampers volume calibration, especially in micron size sampling. Notably, for paper-based immunoassays, saliva's macromolecules (i.e., mucin), human cells (e.g., squamous epithelial cells, white and red cells), and microorganisms (i.e. viruses, bacteria, and fungi) can clog the membrane, reduce or stop the flow rate, and develop a strong background.

To address multiplexing, we provide a vertical flow biosensor (VFB) that we initially designed for an at-microgravity detection kit implemented at the international space station (ISS). This VFB functions solely by capillary force, against gravity, and with the lowest chance of user exposure to any solution and chemical reagents. Its capability to work with an extensive range in volumes of the specimen tremendously increases the sensitivity and accuracy for earlier detection and at-home applications.

A mucus removal reagent (MRR) can be used to address the saliva viscosity and mucus interferences. MRR can remove mucus and reduce viscosity in less than two minutes by forming an aerogel floating on the aqueous solution; the MRR is an elegant solution that involves adding the reagent to saliva, vigorously mixing by hand for a few seconds, and incubating at room temperature for 1 min.

To overcome the costly setup of isothermal amplification, we use a robust chemical heat block for N-gene amplification. The chemical heaters can be used for a low-cost and power-free isothermal amplification; exothermic reactions both in the liquid and solid phases can provide an appropriate source of heat. Preventing chemical exposure to the end-user, controlling the temperature in the desired range, and re-useability of the instrument are the main challenges for satisfying FDA regulation. Several hand warmer pads already available in the market, approved in terms of safety, can be used as reliable sources of heat. Recently, we established a low-cost, user-friendly, and power-less isolation box to provide an accurate and reproducible temperature range of 38-42° C. desired for one-step isothermal amplification (US 2022/0001378 “Smart Storage Container for Health Logistics”).

Considering the typical volume of a saliva sample (about 0.5 mL to 1 mL) and its noninvasive sample collection together with the simplicity of sample preparation by MRR, multiplexing, and flexibility of VFB in handling large volumes of the biological sample, and powerless heat block for isothermal amplification, we propose that VFB will provide a more accurate rapid antibody and N-gene detection test from saliva specimens. Such a multi-functional platform is suitable for detecting active infection, past infection, level of immunization, eligible donors for convalescent plasma, and people at risk, all in one package. At the same time, the integration of VFI from self-collection of the sample to smartphone readout can provide a rapid POC test with the capability of safe monitoring by healthcare providers and experts, which will significantly reduce the risk of exposure to infection.

In this example we: (1) Optimize the one-step isothermal amplification method and integrate the purification step into the device; (2) characterize and optimize VFB for detection of COVID antibodies and N gene; (3) evaluate sensitivity and specificity of the assays. The VFB provides a simple, sensitive multiplex sensor platform for monitoring COVID infection and immune response.

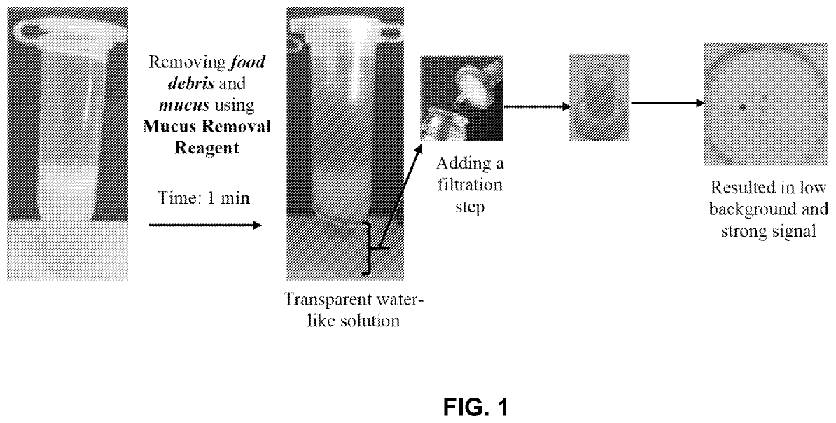

Mucus Removal Reagent (MRR): We provide a polymeric-based solution that can interact with mucin and makes a Floating-Gel on top of the saliva sample (FIG. 1—see first step “Removing food debris and mucus using MRR (time about 1 min)). The initial study shows that big debris and food residues can also be trapped in the Floating-Gel, making a lower particle count in the aqueous phase. There is a direct relation between membrane pore size and signal intensity in vertical flow immunoassays: the smaller the pore size is, the higher the signal intensity is. In the other stream, reduction of membrane's pore size causes clogging of membrane and assay failure. We overcome this issue by adding a filtration step; the filtration of the biological sample just before running the assay can help to remove the background and prevent the membrane's clogging even using 0.2 μm NC membrane (FIG. 1—middle panel “Adding a filtration step”).

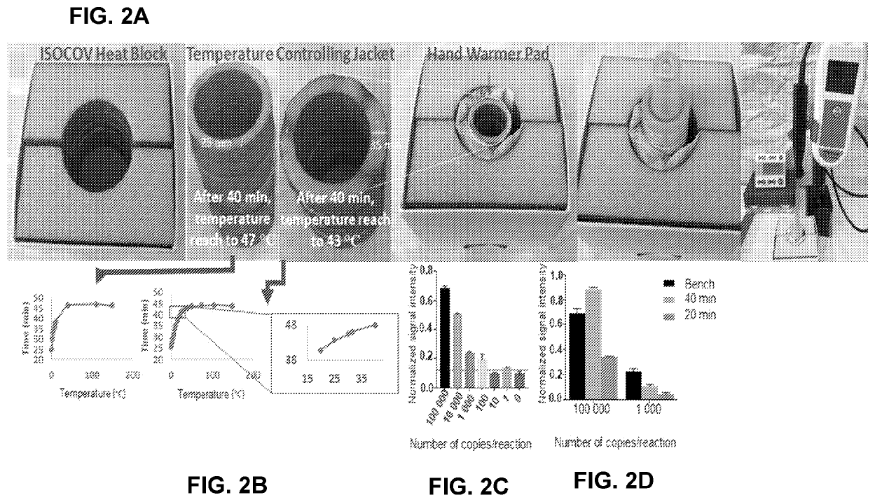

Isothermal Amplification of N gene: The chemical heaters can be used to develop a low-cost and power-free Isothermal Heat Box (FIG. 2A), but safety of these chemical heaters is the main concern to pass the FDA regulation. Several hand warmer products that are already available in the market and approved in terms of safety are reliable sources of heat for power-free and low-cost RPA, but their temperature range (55 to 70° C.) is too high to use in our one-step amplification method. Recently, we have established a low-cost, user-friendly, and power-less isolation box to provide a tunable temperature range of 38-42° C. desired for one-step isothermal amplification. In the preliminary study, LoD for benchtop one-step isothermal amplification using SARS-COV2 RNA spiked in artificial saliva was 10 copies/μL (FIG. 2D), and amplification for 40 min using ISOCOV setup was as efficient as benchtop setup (FIG. 2C). The current design of the heat-block needs 20 min to reach the desired temperature, and more optimization needs to reduce this gap time. The tunability of temperature may include sleeve thickness (FIG. 2B).

Vertical Flow Biosensor (VFB): High multiplexing capability, prevention of Hook effect, handling large sample volume, and fast sensing response make VFBs an attractive choice of POC tests. There are two major types of VFBs categorized as passive and active based on the source of flow force; active VFBs use actuators to run the fluid through the membrane, whereas passive VFBs rely just on capillary force. Among these two, the passive one is more compatible with at-home and low-resource settings, avoiding the complexity, expense, and power control associated with actuators.

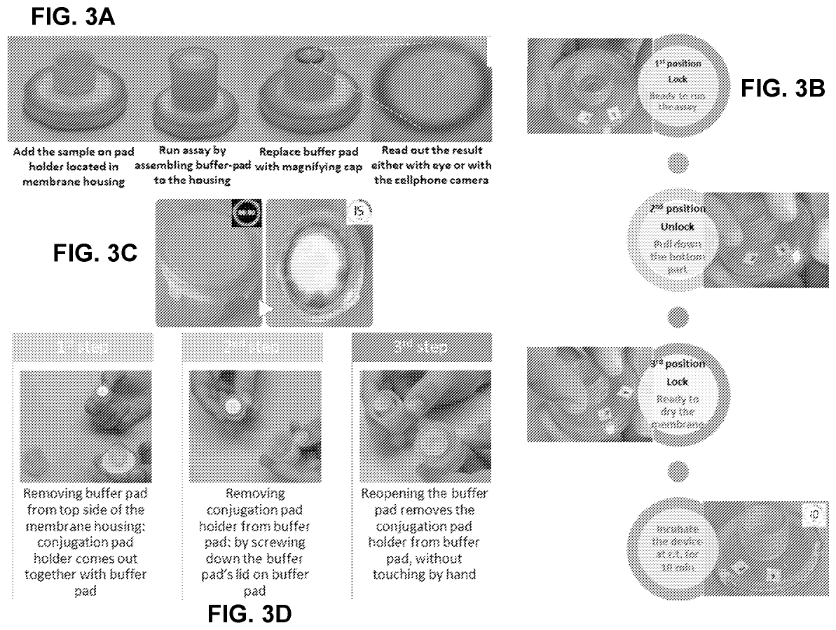

Although simplicity, low-cost, instrument-free setting, rapidness, accuracy, and precision are necessary for such settings, they are not enough to guarantee the success of at- home devices. The safety of end-users and their risk of exposure to chemicals and biohazards should be considered. FIG. 3A is a representative example of an all-capillary force deriving device, including configured for use in zero gravity environment during a crewed flight for a 3 days mission onboard of Space X Dragon spacecraft. Such a mission requires all the necessary criteria similar to consumer-based product with simple use and minimum exposure of user to chemical and biological hazards. Such an embodiment for use as a remote at-home device, may include the following components:

Membrane housing: In addition to the membrane, the housing contains an absorption pad to absorb fluid from the membrane. The preliminary study indicates stronger signals for dried membranes than the wet membrane after finishing the assay. Therefore, adding a short drying step is important in terms of assay sensitivity. At the end of the assay, the absorption pad is wet and prohibits membrane drying. We developed a simple reposition mechanism (FIG. 3B) for the absorption pad to detach it from the membrane without exposing the user to the membrane or absorption pad. A water indicator ring is added at the edge of the absorption pad to calibrate it for stopping the assay after passing a certain amount of buffer (FIG. 3C).

Buffer Pad Capsule: To use in space, assay buffer is stored in a sponge called buffer pad to run the assay buffer through the NC membrane into the absorption pan in the microgravity environment. Later we discovered that this setting could run the assay against gravity, and flow rate and assay time can be controlled by changing properties of sponge (i.e., density, pore size, and hydrophobicity of sponge), which can affect signal intensity (FIG. 4A). Furthermore, the safety mechanism designed for removing pad holder (FIG. 3D) and storing assay buffer in sponge minimize direct contact with chemicals and contaminated parts, making the design ideal for at-home applications.

Pad holder: As mentioned in the sample prep session, a low flow rate of fluid, small pore size of NC membrane (0.2 μm), and nonspecific absorption of non-targeted molecules cause strong background and membrane clogging in assay failure. We showed that filtration can help. To integrate the filtration step, capture antibody-target complexation, and directing-flow pads to the membrane surface into our device (FIG. 4B), we stacked a sample pad, a polyethersulfone membrane (0.2 or 0.45 μm), conjugation pad, retarding pad, and flow directing pad.

Magnifying cap: Magnifying cap contains a low-cost 4× lens for a simple readout of the result. The result can be read out either by eye or by capturing images using a smartphone and analyzing the data by an app. While reading the data by eye makes the device the best choice for fast qualifying tests at low resource settings and catastrophic conditions, simplicity of imaging by putting the phone camera directly on magnifying cap makes the device appropriate for semi-quantitative and even quantitative analyses.

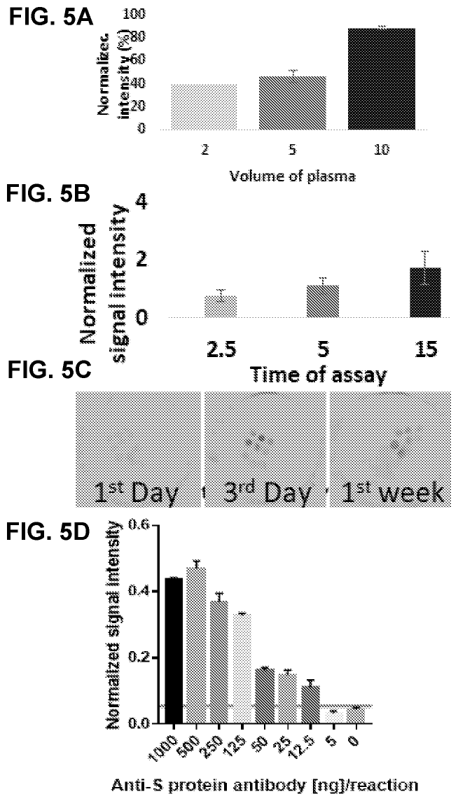

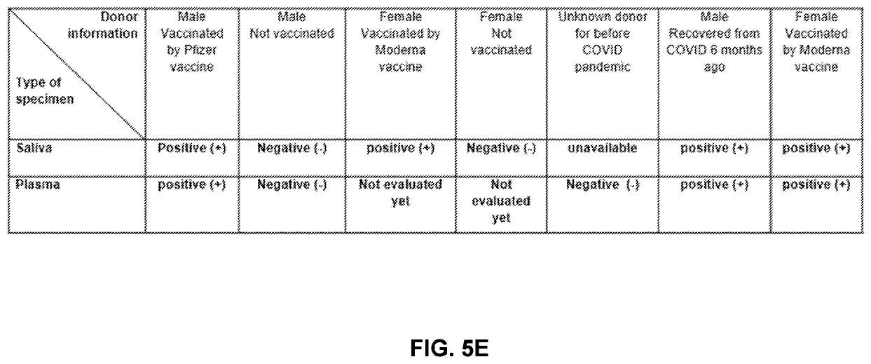

Assay Development: The functionality of the device was evaluated by detecting human SARA-COV2 total antibodies as a low concentration biomarker and total IgM and CRP as a high concentration biomarker. CRP is one of the valuable inflammation biomarkers. However, its high concentration in plasma (less than 3 μg/mL for normal conditions) hampers its detection and quantification in POC set up by the Hook effect. We were able to detect CRP directly from plasma (2-10 μL) without any dilution or sample preparation (FIG. 5A). A wide range of concentrations (1-20 μg/ml of recombinant human CRP) is detectable in our setup at 2.5 min (FIG. 5B). Our preliminary study on stability shows detection antibodies immobilized on the conjugation pad stay functional for more than 1 week (FIG. 5C).

Using recombinant S-protein antibody, we detect as small as 12.5 ng/reaction of antibody spiked in artificial saliva (FIG. 5D). LoD of our assay increased to 83.5 ng/reaction by spiking the antibody into the real saliva. In the preliminary study of SARA-COV2 total antibodies, we were able to detect antibodies both in saliva and plasma in 30-45 min; volunteers who received Pfizer and Moderna vaccines or recovered from COVID-19 showed antibodies in both saliva and plasma. The test was negative for all non-vaccinated/infected persons (FIG. 5E). MRR was used to remove mucus before running the assay with saliva, indicating MRR has no significant interference with the assay. The accordance of saliva's and plasma's results reveals the saliva test's accuracy as a noninvasive antibody test. To the best of our knowledge, there is no saliva-based COVID-SARS-2 antibodies test in the market.

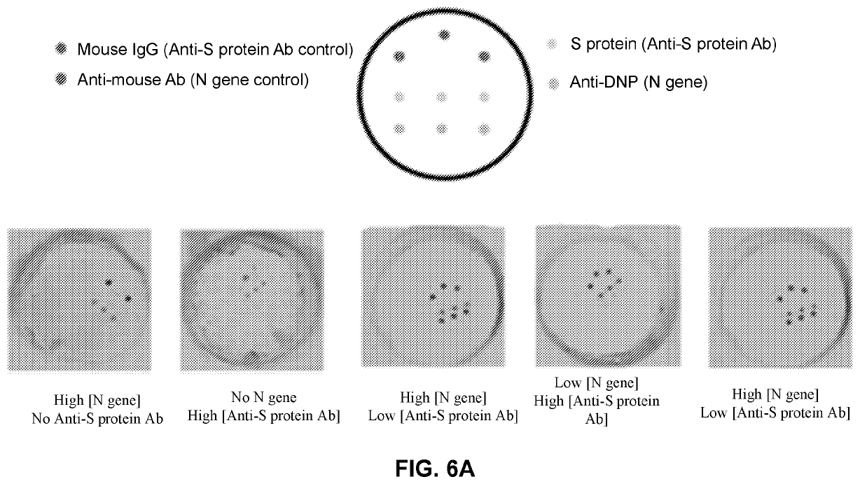

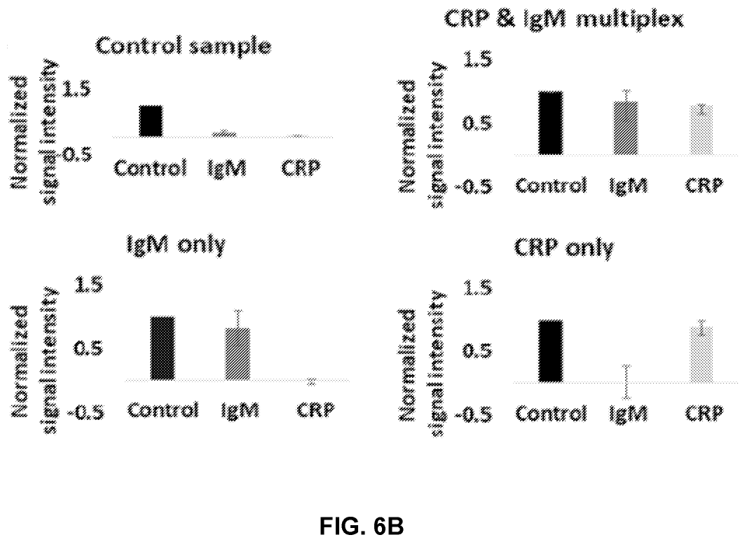

Multiplexing Capability: To investigate the multiplexing capability of the device, we detect N-gene and COVID-SARS-2 antibodies on multiplex assay (FIG. 6A). We could also detect IgM and CRP on the multiplex membrane successfully (FIG. 6B). No significant nonspecific interaction or cross-reaction was observed in the assay, demonstrating a versatile capability of the device in multiplexing using different analytes from genes to proteins.

Mucus Removal Reagent (MRR) Optimization: According to preliminary data, different molecular weights of the polymer and substitution degree of the functional group will be applied to optimize mucus removal and minimized undesired interaction with the immunoassays. Polymer structure and substitution degree will be analyzed by FTIR, HNMR, and C-NMR spectroscopies. Viscosity, particle counts, particle size, and capillary flow rate of treated samples will be evaluated by a viscometer, differentiated light scattering (DLS), and recording video of capillary flow on a paper strip. The effect of sample preparation on the biomarker concentration will be assessed by ELISA and measuring the recovery percentage of spiked antibodies in real saliva.

Isothermal Heat Box and Isothermal Amplification: We will apply foams with different materials and porosity to optimize the heat box. The heat transfer behavior will be optimized using computational modeling in CAMSOL and experimental data to reduce the temperature ramp-up phase and stabilize the temperature in the desired range.

Removing excess amounts of labeled primers after amplification can be addressed to significantly improve the sensitivity of the detection assay. The extra amount of labeled primers in the detection step drops the assay's sensitivity by increasing the number of false-negative and false-positive results. Affinity column purification is well established for benchtop amplification setups but needs centrifugation that is not compatible with the instrument-free approach in POC tests. Magnetic bead technology can be used alternatively; preliminary data confirmed the assay's sensitivity over non-purified samples. However, this approach has several steps, and integrating this technology into the at-home test is challenging. We can use cap-on-cap, and Lego game approaches to overcome this difficulty. In a case of failure by magnetic bead technology, we can implement enzymes or chemical reagents for purification as a backup.

Device Design: As mentioned, we provide proof of principle as to the functionality of the VFB, MRR, and Heat Box. However, we can further integrate sample preparation, amplification, and VFB.

### Sensitivity Enhancement—Antibody Test

To optimize the assay and enhance sensitivity, several target proteins (e.g., S-protein, N protein, and their different domains) will be evaluated. If different vaccines target one of the molecules better than others, our multiplexing capability can help us design a test for detection of all targeted proteins of the SARS-COV-2 virus. Then the effect of detection antibodies concentration, time of assay, NC membrane pore size, hydrophobicity, and porosity of sponge used in buffer pad will be optimized using design of experiment. According to the current FDA guideline for SARS-COV-2 antibody test EUA, we will evaluate more than 75 negative samples together with all our positive samples to waive cross-reaction study. If the FDA guidance changes to regular procedure, we can evaluate the cross-reactivity of our assay with various antibodies, including the antibodies of TABLE 2.

TABLE 2
List of antibodies considered in cross-reactivity study:

anti-influenza A (IgG and IgM)
anti-influenza B (IgG and IgM)
anti-HCV (IgG and IgM)
anti-HBV (IgG and IgM)
anti-Haemophilus influenzae (IgG and IgM)
anti-229E (alpha coronavirus)
anti-NL63 (alpha coronavirus)
anti-OC43 (beta coronavirus)
anti-HKU1 (beta coronavirus)
ANA
anti-respiratory syncytial virus (IgG and IgM)
anti-HIV

### Example 2: Point-of-Care (POC) assays as a diagnostic platform

Two top national security priorities are developing pre-exposure radioprotectors and post-exposure therapeutic agents. The Public Health Emergency Medical Countermeasures Enterprise also listed radiological and nuclear agents as a top medical countermeasure priority. The earthquake and related tsunami off the coast of Japan show that natural disasters can lead to mass radiation exposure. At this point, the only known therapy for severe radiation exposure is bone-marrow transplantation. This therapy is complex, treats only bone marrow failure, and requires appropriate donors. Transplantation is also risky, unaffordable on a large scale, and time-consuming, thus making it impractical for mass intervention. Furthermore, there is no available therapy for multiple-organ system damage in humans exposed to whole-body radiation injury.

We have identified a new therapy to treat radiation exposure. We have shown in the murine system that mesenchymal stem cells (MSCs) overexpressing extracellular superoxide dismutase (ECSOD), i.e. ECSOD-MSCs, have a significant mitigative effect against otherwise lethal radiation injury. One dose of ECSOD-MSCs given 24 hours after radiation exposure improved survival from 10% to 52% in irradiated mice. Therefore, with permission for clinical use, this innovation will enable strategic stockpiling of ECSOD-MSCs as a medical countermeasure against radiation exposure.

We will advance the therapy characterization as we will develop, optimize, and validate our technology platforms in a murine model, prove our concept in non-human primates, and demonstrate the safety of our therapy in cancer patients undergoing palliative radiation therapy in a Phase I, FDA-approved clinical trial. We will (1) establish the safety profile, optimal dose, timing, frequency, and protocol of the new therapy in an appropriate murine model setting; (2) generate toxicity data to establish the therapy's readiness for human use; (3) begin the work of validating the new therapy in tumor models prior to a Phase I trial in humans; and (4) start a small scale GMP production.

Our therapy is novel and significantly advanced compared to other work in the field. We have demonstrated greater effectiveness and our therapy can be administered post-exposure, which is the most likely application for a radiation-exposure medical countermeasure. We have also demonstrated that ECSOD-MSCs improve survival, extend lifespan, slow cataract formation, and may prevent tumor formation in irradiated mice. Moreover, we utilized the only form of ECSOD-MSCs that is effective in the extracellular context, therefore yielding a systemic effect.

Our development program addresses an important public health threat and aligns with BARDA's mission to provide medical countermeasures. A therapeutic modality for the at-risk population following radiation exposure is relevant scientifically and as a national security priority. Targeted therapeutic intervention is also relevant to the goals and priorities currently stated by NIH and DHHS and will further radiation biology and stem cell research. Our program will be a first comprehensive and effective stem cell-based gene therapy medical countermeasure that can be used after a radiological or nuclear event. Once fully developed, our program will provide the capability to rescue more than 50% of the exposed population with a single-dose injection.

Point-of-care (POC) assays, including those provided herein, are essential diagnostic platforms that allow fast health monitoring and disease testing with minimal equipment. Their use has shown to improve both clinical and economic outcome by, for example, allowing faster decision, starting treatment earlier, reducing use of staff, equipment, and hospital admissions. In a context of pandemic such as the COVID-19, their use would drastically improve public health management and outcome. Effort is provided around a multiplexed detection of four tier 1 biothreat agents using a Vertical Flow paper-based Immunoassay (VFI) (US 2021/0199651 “Vertical Flow Molecular Assay Apparatus”). Within a miniaturized “syringe-like” device, we have demonstrated the functionality and advantage of the VFI platform with an improved limit of detection >25× vs. standard lateral flow assay for the detection of the bio-threat pathogen Burkholderia pseudomallei, showing the ability of VFP to detect microbial antigen or more generally proteins [Peng et al. 2019]. Interestingly, we then adapted this platform to allow also the detection of nucleic acids. Indeed, by using the same immunodetection approach, we developed an assay to amplify and quantify RNA levels of two biodosimetry and one housekeeping genes in order to monitor absorbed radiation dose from blood in low-resource environment such as during space flights. With the sudden COVID-19 pandemic, we rapidly adapted this VFI platform to an even more compact format that does not require any power supply or any heavy instrumental handling, and that can be performed without the need of medical facilities, in less than 1 hour, from sample collection to data analysis (see Example 1). Based on this new design, we pursued the VFI development for space applications, showing its capacity to work in microgravity environment to provide multiplex data analysis from fingerstick collection.

### Technical Approach

Our new simplified platform can provide an efficient powerless and cost-efficient point-of-care assay able to detect both SARS-COV-2 particles and/or its associated immune response from saliva samples in remote environment (OMTest). For this, we first optimize the most advanced feature of our platform (SARS-COV-2 antibodies detection) for a rapid Emergency Use Authorization (EUA) application at the Food and Drug Administration (FDA) (Phase one) before developing the platform for a fully integrated SARS-COV-2 gene detection while optimizing manufacturing and packaging of the device. Finally, we merge both applications in a single device to provide a complete multiplex point-of-care bioassay.

Example 1 demonstrates that the VFI can detect Ig antibodies against SARS-CoV-2 Spike glycoprotein in human saliva. For this, we specifically developed a polymeric-based solution that can interact with mucin to decrease saliva viscosity not only by binding mucin but also by trapping debris and food residues. By integrating this step in our workflow, and as a proof of concept, the VFI was able to rapidly detect, up to 85 ng of recombinant anti-SARS-COV-2 Spike(S) glycoprotein antibody spiked in real saliva. Interestingly, this approach has also been able to accurately detect immunized persons (i.e. vaccinated or COVID-19-recovering patients) while displaying negative results for non-vaccinated or-infected individuals. In phase one, the device is validated and developed in the context of a saliva-based point-of-care COVID-19 antibodies test.

Performance evaluation of VFI platform to detect SARS-COV-2 humoral response using saliva samples: We determine on a large number of samples (>100) the specificity and sensitivity of the OMTest. Sensitivity will be assessed by saliva samples collected both from SARS-COV-2 vaccinated individuals or patients infected by SARS-COV-2 and confirmed by qRT-PCR. Specificity will be assessed using saliva samples collected before SARS-COV-2 was known to have circulated. Samples from patients having other respiratory infection but known to be truly negative (never vaccinated, never infected) could also be used. Optimization of the assay parameters that include mucus removal reagent composition, antibodies concentration, time of assay, membrane pore size, hydrophobicity and porosity of the pad, assay buffer composition will also be performed to reach the maximum sensitivity while minimizing non-specific binding. An objective is to demonstrate that the OMTest can achieve >90% in sensitivity and >95% in specificity in order to satisfy FDA guidelines.

Development of a OMTest discriminating SARS-COV-2 IgG and IgM response: Our current data is obtained using a gold nanoparticle conjugated anti-human Ig as detection antibody that targets independently IgA, IgG and IgM. We can further improve the test to differentiate specifically IgM and IgG responses.

Demonstrate the feasibility of OMTest to detect IgM and IgG response on independent membranes: In a first objective, detection antibody will be replaced by either anti human IgM or anti human IgG. Real saliva samples collected before the pandemic will be spiked at different concentration either with human IgM anti-SARS-COV-2 Spike or human IgG anti-SARS-COV-2 Spike antibody and detected using anti-human IgM or IgG on a recombinant SARS-COV-2 S-protein coated membrane to assess sensitivity and specificity. Alternatively, SARS-COV-2 antibodies positive saliva samples depleted either for human anti-IgM or IgG can be used for validation.

Develop a multiplex platform able to perform simultaneous detection of IgM and IgG response: Two strategies could be used for this objective. The first strategy will explore a different design in order to split the sample to be detected on two different membranes, coated with either anti-human IgM or IgG. A second strategy will explore a different version of the bioassay where a recombinant SARS-COV-2 S-protein is conjugated to gold nanoparticles, interact with the biological sample that will be then recognized by anti-human IgM and IgG antibodies coated on a specific part of a single membrane.

As highlighted previously, using the same immunodetection approach, we demonstrated that our platform could also detect and quantify gene expression. Therefore, we propose to develop a duplex assay where immune response (detection of anti-SARS-COV-2 Spike protein antibodies) and viral particles concentration (detected by presence of viral genome) could be both detected on the same membrane using saliva sample.

Using a benchtop sample lysis and purification protocols, our preliminary data showed that VFI membrane could be used to detect at least 100 copies of viral RNA/reaction (10 copies/μL) after isothermal amplification. In order to perform gene detection on the OMTest, the protocol for the three major steps (i.e. sample preparation, amplification, purification) is required to be adapted to avoid powered large instruments and harsh treatments such as high temperature, sonication, or high centrifugation.

Different lysis protocols will be tested including low temperature treatment in complement to chemical lysis. Treatment will be optimized in order to also preserve the sample for the subsequent immune response analysis and to be compatible with the mucus removal reagent.

### Development and Optimization of the One-Step Isothermal Amplification

Multiplex isothermal amplification: As mentioned previously we already successfully detected SARS-COV-2 N gene using VFI membrane. In order to improve the assay, we exploit the multiplex capability of the VFI by adding the detection of one more target (e.g. additional primer pair for N gene) or by integrating an internal control targeting RNase P (extraction control) as recommended by FDA.

Powerless isothermal amplification: We developed a one-step isothermal amplification benchtop protocol that uses a constant and unique low temperature (˜42° C.) to perform both reverse transcription and amplification. Therefore, we also have established a low-cost, user-friendly, and power-less isolation box to provide a tunable temperature range of 38-42° C. using hand warmer products that are already available in the market and approved in terms of safety. Our data showed that an amplification for 40 min using this setup was as efficient as benchtop protocol to detect 100 copies of viral RNA. The current configuration of the heat-block needs 20 min to reach the desired temperature and the design requires modifications to be fully compatible for the VFI integration (i.e. weight, size, etc.), so additional optimization will be performed to address these gaps.

Purification protocol: The simple nucleic assay included in the VFI requires a purification step at the end of the amplification in order to both reduce background and improve sensitivity. Current protocol employs column-based purification using high centrifugation speed. However, we already showed that this protocol could be replaced by a magnetic bead approach.

The three major steps are integrated in the VFI platform in order to provide the first complete point-of-care saliva assay to detect SARS-COV-2.

Limit of detection is assessed using blinded saliva samples spiked using different SARS-COV-2 viral particles concentration. Specificity is assessed by testing cross-reactivity with other microorganisms including adenovirus 5 and 11, other coronaviruses (e.g. SARS-CoV-1, MERS, etc.), influenza A and B, rhinovirus 61, parainfluenza 1, 2, 3 and 4b, Streptococcus pneumoniae, Mycobacterium tuberculosis, etc. Clinical evaluation will be performed by screening double-blinded infected patient saliva samples and determining positive and negative percent agreement.

In order to promote a more advanced and unique test, we integrate the SARS-CoV-2 N-gene detection along with the immune response. Our preliminary data already shows the ability of our approach to detect simultaneously on the same membrane both amplified N-gene plasmid and recombinant anti-SARS-COV-2 S protein antibody spiked in artificial saliva. Results show that there is no cross-interaction between the different reagents and that the same membrane can detect both products together or independently without significant background (i.e. minimizing risk for false positive).

### Example 3: Space-Based Paper Microfluidic Vertical Flow Assay for Rapid Multiplex Diagnostics (VFD) in COVID-19 Pandemic Response and Recovery

There is a need for rapid test kits that will allow analysis closer to the point-of-care, while offering results in minutes rather than days, including for COVID-19. To date, not everyone who has signs of COVID-19 disease may be tested. Another challenge can be the low supply of some of the reagents and components needed to run tests. Furthermore, patients who test negative are presumed to be so, but current nasopharyngeal PCR swab tests are only 70% accurate. There is also missing information on how the disease develops, so serological testing that measure antibodies in blood, will tell whether a person has been recently infected with SARS-COV-2 and possibly recovered. Other CRISPR-based detection platforms are targeting the genes of the viral surface proteins. Here, we propose exploiting the unique features of the Vertical Flow Diagnostics (VFD) multiplexing and universal detection of both proteins and nucleic acids. The development of new synthetic antibodies panels that can identify and target against various proteins of the COVID virus (SARS-COV-2) combined with RNA biomarkers will provide rapid Point-of-care detection and critical complementary information about how the virus spreads, but also about the case fatality rate and the fraction of infections that result in death. It will also guide the necessary safe screening of employees to keep workers working. We have developed a paper-based Vertical Flow Diagnostics (VFD) that can perform measurements under the requisite parameters identified by the FDA and CDC guidelines. In this example, the plan for the proposed targeted product profile (TPP), with a technological readiness level (TRL) of 5-6, comprises the design and validation of the enhanced VFD diagnostics, readily scalable and adaptable to reagents production and mass manufacturing of devices, with specific panels for SARS-COV-2 exposure and, as desired, panels against other infectious agents, complementary to the multiplexing functionality for the detection of other bio-signatures of Space Health applications.

Innovations and components of this Space health technology for Covid19 diagnostics include:
(1) Develop a multiplex antibody based SARS-COV-2 detection assay configured to the VFD platform technology developed for Space application to meet the Point-of-Need requirements in COVID 19 pandemic response and recovery missions: Multiple approaches will be taken to identify antibodies that will function in the VFD assay. Since SARS-COV-2 is a newly discovered virus we will first analyze available antibodies that bind to SARS-COV-1 for reactivity with SARS-COV-2. A number of these antibodies are becoming available; some have already been shown to bind to SARS-COV-2. Adding to the currently available commercial antibodies, we will use our Antibody Facility, which has a library of over 17,000 antibody sequences, which include 47 recombinant antibodies against the SARS-COV-1 S protein, 56 against the MERS S protein, and 4 against the SARS N protein. Based on homology analysis between SARS-COV-1 & 2 it is highly likely that a number of these antibodies will be cross-reactive. New “nanobodies” will also be identified directly against SARS-COV-2 proteins. Furthermore, we have immunized mice with recombinant forms of the SARS-COV-2 S1 and S2 spike proteins along with nucleoprotein (N) protein, so we will access their sequences and produce them using recombinant production.
(2) Validate enhanced analytical sensitivity or limit of detection (LOD) within control buffers and relevant matrix (e.g. Nasopharyngeal or oropharyngeal 3D-printed new swab design to improve collection and release of samples re-suspended in assay running buffer, etc.) samples spiked with recombinant viral antigens (pg/ml determination) and purified viral preparations (pfu/ml or genome equivalents/ml determination). Multiple antigens will be produced and utilized for spiking experiments including, recombinant SARS-CoV-2 proteins (S1, S2 & N), virus like particles (VLPs), and inactivated SARS-COV-2 viral preparations.
(3) Confirm analytical reactivity and specificity in the VFD multiplex format with viral species, near neighbor species and species of virus that produce similar symptoms in patients. A number of SARS-COV-2 viral preparations and clinical samples are available for this project. A heat-inactivated SARS-COV-2 isolate (SARS-Related Coronavirus 2, Isolate USA-WA1/2020) has been ordered from BEI Resources in addition to access to over 10's of clinical isolates tested positive and counting at both the Nevada State Public Health Laboratory. Similar samples will also be made available to our team from the laboratories at TGen and the Arizona Department of Health and Services. The genomes of these SARS-CoV-2 clinical isolates are currently being sequenced by TGen and the Nevada Genomics Center; this will provide valuable phylogenetic reactivity information. In addition, many live/inactivated isolates from around the US and world are available for reactivity testing. Live SARS-COV-2 isolates could be tested within the BSL3 laboratory (Aucoin) at UNR MED. Specificity testing will include near neighbor SARS-COV-1 and MERS inactivated isolates acquired from BEI resources and collaborators. In addition, the AuCoin laboratory possesses a large panel of live/inactivated bacteria, virus and fungal isolates. Pathogens that exhibit similar clinical features as SARS-COV-2 will be of particular importance.
(4) Optimize the VFD device with reagents, automated smartphone-based imager with software.
(5) Scale-up prototyping of devices accordingly to compliant design control processes at industry partners.
(6) As part of the process, a Covid-19 test system validates the assay kits, and provides optimization and performance evaluation studies to confirm that the VFD meets pre-defined performance specifications for SARS-COV-2 detection and PON system functionality.
(7) For both clinical sensitivity and specificity a target performance milestone is set at ≥ 90%.
(8) Based on feedback, the system design can be refined.

### Example 4: A Rapid Powerless Multiplex Self-Diagnostics

Development and validation of a safe, user-friendly, comprehensive and truly home diagnostics device and kit (OMTest) for the rapid multiplex detection of emerging infections. The device integrates novel, yet proven, technologies to provide rapid parallel analytic capabilities, including both proteins and nucleic acids assay platforms. This device and kit is completely self-contained, preventing exposure of users to biohazards from sample self-collection to processing and final data output. The device and associated test kit do not require any electrical battery or wall power, while data is obtained by optical read-out, such as by visual inspection or using a cybersecure smartphone App.

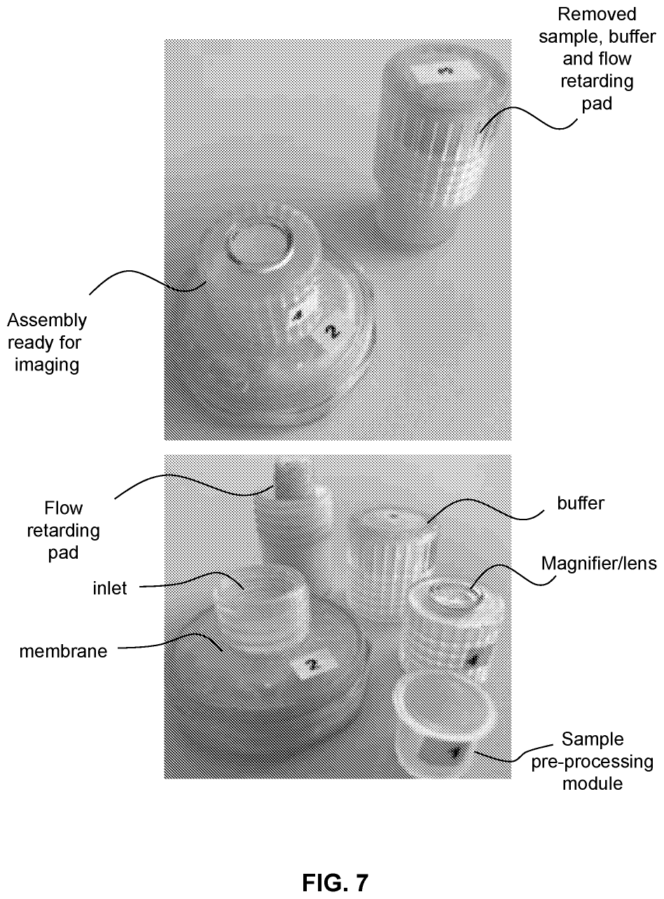

Effort includes: 1. Automation of all aspects of saliva/blood self-collection & prep; 2. Integration of reagents into sequence of “tube-and-cap” devices (FIG. 7); 3. Multiplexed antigens analysis for SARS-Cov2+host antibodies; 4. Validation of clinical samples accordingly to FDA EUA guidelines; 5. App development for image analysis and cloud communication; 6. Validation of OMTest with CLIA-lab partner(s).

Benefits: Current commercial technologies are not capable of comprehensive-multiplex analysis of SARS-COV2 infection from non-invasive saliva or finger-prick blood samples to be performed safely by a consumer at home or in a remote low resource setting. The Vertical Flow Assay capacity provides a rapid, sensitive and highly multiplexed infectious agents platform, not previously available. The sequence of tubes and caps assembly provides a “safe and simple” workflow analysis, allowing for a survey of genes, antigens and antibodies for many targeted pathogens, including coronovirus emerging infections, at very affordable cost enabled by testing anytime, anywhere, without the need of any instrument or electrical power source.

Challenges: As the project plan includes the use of multiple assay chemistry technologies, the challenges are primarily: A) the integration of these technologies; B) their packaging and shelf-life; and C) the regulatory requirements to deploy into the consumer market.

### Example 5: VFI Biodosimetry Summary

We have developed a vertical flow immunoassay (VFI) to detect biothreat pathogens (Peng et al. 2019. Devadhasan et al. 2021). Using a nanoporous nitrocellulose membrane encapsulated in a stainless-steel filter holder, the sample is actively pushed through the membrane, which is pre-functionalized with capture antibody, and where a sandwich immunoassay is formed. The colorimetric signal generated by the presence of gold nanoparticles then reflects the target antigens. Results showed that by optimizing flow speed and membrane pore size, the platform could detect Burkholderia pseudomallei at a concentration up to 0.02 ng/mL, making the VFI much more sensitive than standard approach such as ELISA (Peng et al. 2019). Recently, the miniaturization of this platform even allowed the detection of a Yersinia pestis biomarker with a sensitivity 10 times better than that of the previous large VFI platform and 80 times over a standard lateral flow configuration (Devadhasan et al. 2021).

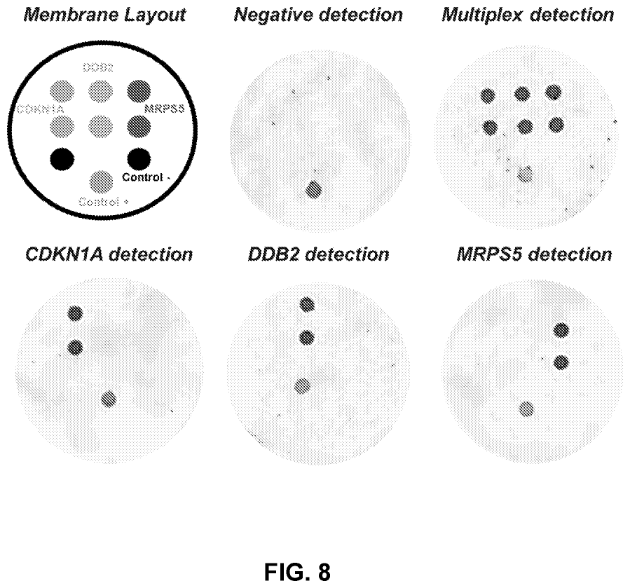

In order to expand the features of this miniaturized platform, we adapted it to allow nucleic acid detection from blood cells and to monitor biodosimetry gene expression and radiation exposure. By using a similar immuno-approach, and labeled specific primers, we designed an assay to amplify and quantify RNA levels using a multiplex membrane. The bioassay showed that it can detect the expression level of two biodosimetry genes (CDKN1A and DDB2) while using a housekeeping gene (MRPS5) for normalization with a high specificity (FIG. 8).

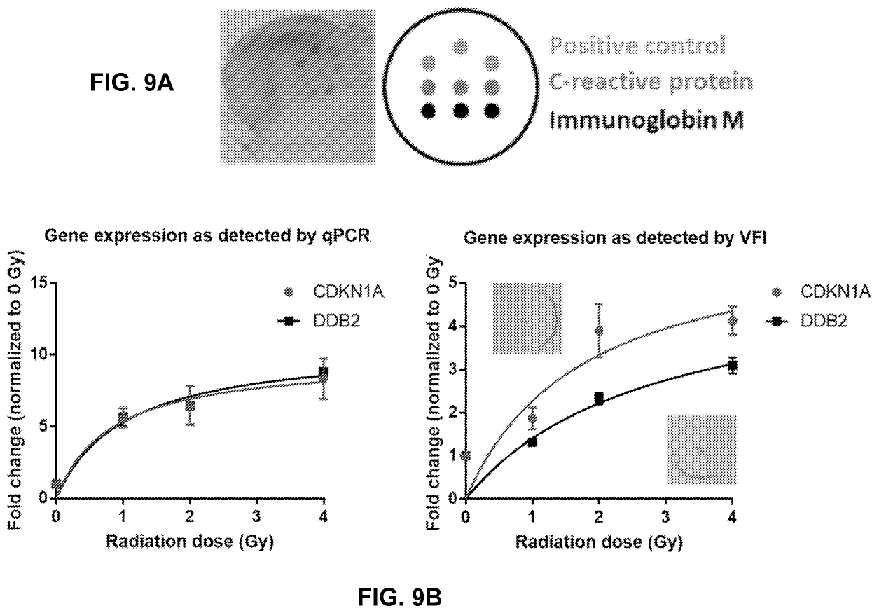

Our preliminary data demonstrates that, similar to qPCR, the VFI platform detects an increase in expression level of these biodosimetry genes in function of the dose (compare FIG. 9A with FIG. 9B), suggesting that the VFI can be used to monitor absorbed radiation dose and, at large, to measure efficiently expression of gene biomarkers.

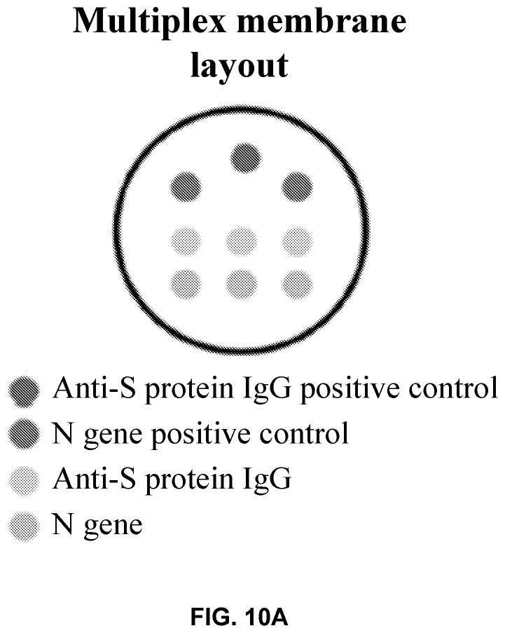

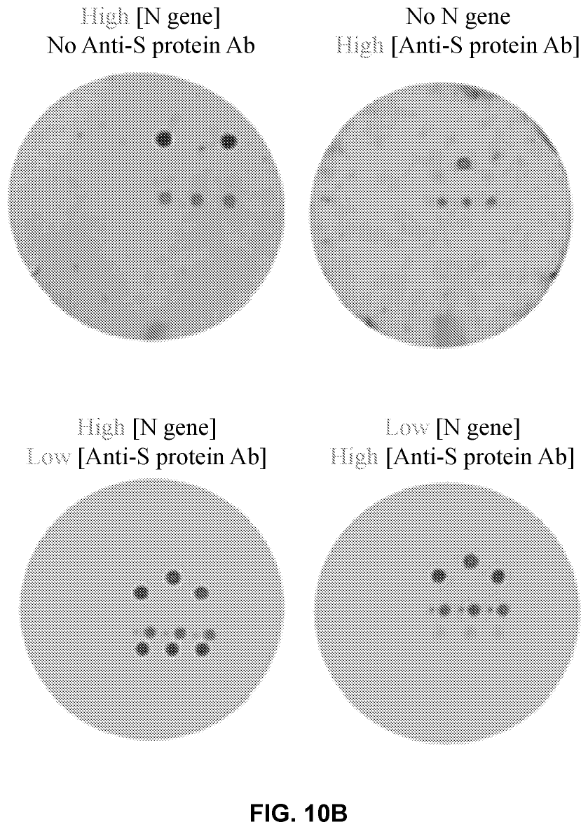

Finally, with the emergence of COVID-19 pandemic, we can combine these main features to demonstrate the capacity of the VFI to detect simultaneously both gene (SARS-CoV-2 N gene to detect viral particles) and protein (human anti-S protein IgG to assess immune response to SARS-COV-2) on the same membrane (FIGS. 10A-10B). This is a proof of principle approach for a single device that can detect biomarkers associated with both a virus and a protein reflecting host immune response. In this manner, status of an individual with respect to viral infection and immune response status, such as from a previous vaccination, is achieved in a safe and straight-forward on-demand home testing system.

Gene and antibodies have been spiked into artificial saliva or plasma (for antibodies only) and samples have been detected using a VFB at a minimal concentration of 10 copies/μL and 12.5 ng/reaction respectively using a device of the instant invention (see, e.g., Example 1; FIG. 4B), also referred herein as the OMTEST™ kit.

The OMTEST™ kit is a versatile platform complemented with its simplicity of use without the need of any electronic instrumentation (e.g. syringe pump or thermocycler) since assay buffer and reagents are contained in absorbing pads and moved by capillary force. Genes amplification is performed in the carton box of the kit with an air-activated hand warmer pad and readout of the assay array dots can be visualized by the naked-eye or using the VeriFAST APP on a smartphone (iPhone). One goal is to optimize the device, and the associated platform required for sample preparation, to perform remotely detection of dosimetry gene from whole blood. The device is also compatible with detection of a panel of biomarkers for major biothreat pathogens, thereby serving as a primary fast screen in an emergency situation.

### Example 6: Targeted Biomarker Panels and Pre-Processing Device for the Rapid Assessment of Radiation Injury in Easily Accessible Biofluids

This example builds upon our metabolomics expertise in untargeted and targeted metabolomics for the generation of highly sensitive and quantitative targeted multiplex assays. The idea behind generating such assays for radiation assessment and radiation injury in easily accessible biofluids (urine, blood, saliva) is to rapidly determine the extent of exposure of an individual and distinguish between the worried well and the exposed individuals that may require medical intervention. Highly quantitative approaches will be undertaken through liquid chromatography tandem mass spectrometry (LC-MS/MS) to quantify each already identified radiation biomarkers in each biofluid. Such instruments are currently used routinely in clinical laboratories, therefore, maximizing the available resources for rapid evaluation of thousands of individuals during an emergency. Based on criteria for sensitivity, high signal-to-noise ratio, low signal suppression from matrix effects, and high fold changes compared to controls or relationships between pairs of metabolites, biosignatures will be assembled and concentrations calculated. The combined biosignature will be developed in a multiplex assay, effectively reducing the time between sample preparation to results. The goal is to demonstrate that this multiplex assay method has the potential to be deployed in the case of an emergency to a pre-determined network of clinical laboratories that can accept and rapidly process a high volume of samples. While the ultimate goal will be for such a panel to be predictive in all cases, even a limited false positive rate would facilitate assessment of radiation injury in a mass casualty scenario: e.g. a 1% false positive rate would reduce the number of individuals needing further evaluation by 100-fold. Additionally, this assay will be flexible as it could be enriched with biomarkers for specificity and radiation quality. This example also further develops pre-processing devices with the intention of stabilizing the sample during transport to a clinical facility. The materials to be fabricated will also aim to enrich the biosignature for the radiation-related metabolites and extract them effectively from small amounts of a biofluid (urine, serum, whole blood, saliva), transported as a stable dry membrane. Assembly of such materials in a 96-well plate will further decrease the sample preparation time and minimize human error associated with sample preparation. Our unique approached to combine LC-MS/MS applications with pre-processing materials will aim to move this technology from the feasibility stage to technology development, satisfying the needs for rapid methods for radiation injury assessment.

Radiation metabolomics is a well-established field in biomarker discovery and in assessing changes associated with metabolic perturbations, whether in tissues or biofluids. We have developed a rich database of radiation biomarkers in urine, blood and saliva and we now plan to develop multiplex assays that are highly quantitative and can serve as the first step towards a deployable assay using commercially based instruments. Additionally, we will further develop membrane-based technologies that will aim to eliminate the pre-processing steps, thereby jump-starting the sample processing step and enrichment for specific classes of metabolites and stabilizing the biosignature during transport.

### Example 7: Point-of-Case Paper-Based Flow Immunoassay for the Detection of FAST-DOSE Markers

In the spectrum of a response to a radiological incident, mass casualty care would impact the medical system drastically, that would necessitate a rapid and accurate triage system for high throughput sorting of victims from children to elderly. It is a challenge for medical responders to triage individuals who are minimally exposed and do not need treatment compared to those who received higher dose radiation and may need immediate treatment. Our long-term objective goal is (1) to identify a radiation dosimetry signature from blood biofluid markers whose quantification is not labor intensive and time consuming and (2) to develop a point-of-care (POC) bioassay to integrate this signature and allow detection in a remote environment by non-trained users. To address this, provided is a biomarker device for high-throughput biodosimetry, designed to rapidly quantify the upregulation of radio-responsive intracellular proteins in blood leukocytes from small volumes of blood for retrospective dose reconstruction. This can be achieved using a VFB, including a paper-based microfluidic point-of-care (POC) vertical flow immunoassay (VFI) that can detect multiple biofluid markers simultaneously, within minutes, and does not require any power supply or any heavy instrumental handling allowing its use by unexperienced users outside medical facilities. A sandwich paper-based immunoassay can be used for the detection of FAST-DOSE proteins using the VFI platform in order to discriminate exposure <2 Gy from ≥2 Gy. In this manner, VFI is integrated with FAST-DOSE biomarkers to discriminate irradiated samples using a simple kit without any specific instrumental equipment and with a simplified workflow accessible for all users in non-clinical setting or at home. These results will have an important positive impact as they provide the foundation for optimization and future large scale VFI manufacturing.

In a nuclear or radiological incident, first responders must quickly and accurately measure radiation exposure among civilians, as medical countermeasures are radiation dose-dependent and time-sensitive. It would be particularly useful to employ a simple point-of-care bioassay kit for the detection and quantification of blood proteins whose level have been linked to the amount of radiation dose exposed to individual, thus providing results within minutes for any users without the need of clinical facilities. The devices and methods provided herein are affordable POC assays that can be used to improve the civilian triage and treatment following a catastrophic event, such as a radiation exposure event. As discussed elsewhere, other applications include assessing whether individuals have been exposed to a contagious agent, such as a virus, bacteria, or a toxic chemical.

Following a large-scale, radiological incident, there is a need for FDA-approved biodosimetry devices and biomarkers with the ability to rapidly determine past radiation exposure with sufficient accuracy for early population triage and medical management. Towards this goal, we are developing the FAST-DOSE (Fluorescent Automated Screening Tool for Dosimetry) device for high-throughput biodosimetry, designed to rapidly quantify the upregulation of radio-responsive intracellular proteins in blood leukocytes from small volumes of blood for retrospective dose reconstruction. The can be implemented via the instant VFB, such as a paper-based microfluidic point-of-care (POC) vertical flow immunoassay (VFI) that can detect within minutes multiple gene or protein biomarkers simultaneously, in different type of biofluids. The device is adapted to remove any power supply and simplified to avoid heavy instrumental handling and protocol, making it a simple POC platform to be used everywhere, even by unexperienced users. As described in other examples herein, the VFB-containing device can be used for the detection of COVID-19 antigens as well as inflammatory and immune plasma proteins.

This example represents another example, a POC bioassay that can rapidly detect and quantify radiation dosimetry markers for triage in case of mass-casualty nuclear/radiation incident. This can be implement in the form of a sandwich immunoassay to detect FAST-DOSE intracellular protein biomarkers that are compatible with a capillary-driven paper-based approach in order to integrate the FAST-DOSE signature in the VFI platform.

The device is designed to rapidly quantify the upregulation of radio-responsive intracellular proteins in blood leukocytes from small volumes of blood for retrospective dose reconstruction after exposure to ionizing radiation [1].

Membrane-based devices are employed for the collection enhancement and preservation of blood-based biomarkers. This has been demonstrated on fabric fibers [2], but is also compatible with paper substrates, including a paper-based microfluidic POC vertical flow immunoassay (VFI) [3,4] that can detect multiple gene or protein biomarkers simultaneously, in biofluids, and does not require any power supply or any heavy instrumental handling (FIG. 7). This device is also useful for the multiplex measurement of radiation dosimetry genes and also for antigens reflected of infection, such as COVID-19 antigens. The device has also been tested in remote and extreme environment during an Inspiration4 mission (SpaceX) to detect inflammatory and immune blood proteins. This example integrates the detection and quantification of a plurality of proteins, such as up to 6 intracellular radiation dosimetry proteins on the VFI device.

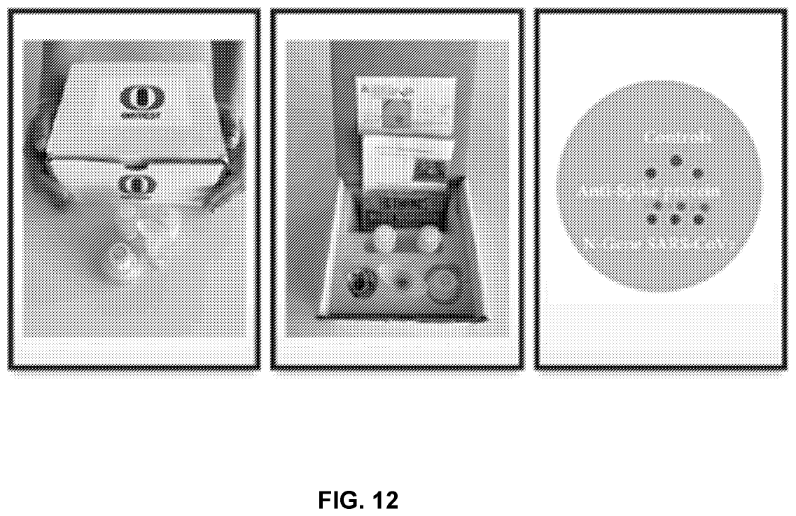

Because of its simplicity of use that does not require access to medical facilities and its data analysis automation through direct naked eye visualization or using a smartphone app (FIG. 15), the device/kit can be easily deployed in the field (FIG. 12), directly used by any non-experienced users, and provide results within minutes, making it an ideal tool for triage in case of mass-casualty nuclear/radiation incident. This example demonstrates the VFB can comprise a sandwich immunoassay to detect FAST-DOSE intracellular protein biomarkers that are compatible with a capillary-driven paper-based and multiplex approach.

Irradiation of blood samples ex vivo from healthy human adult subjects.

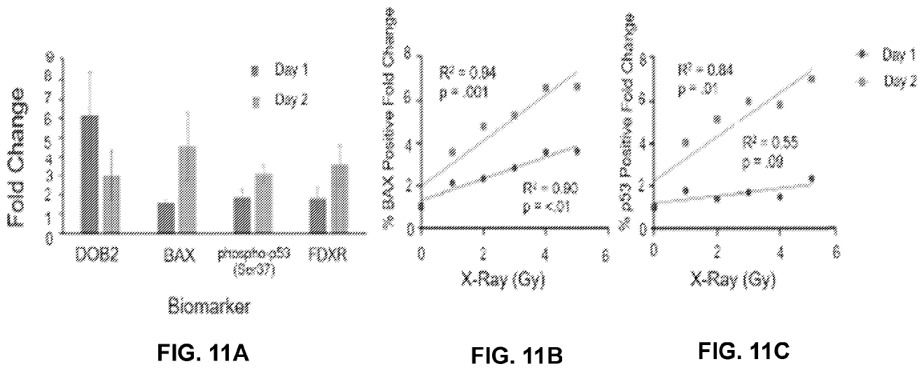

A FAST-DOSE protein biomarker panel includes FDXR, ACTNI, DDB2, BAX, phospho-p53 (p53), and TSPYL2, and is tested for sensitivity, variability, and reproducibility using the human blood ex vivo model. The immunolabeling protocol is optimized to amplify biomarker signal in specific leukocyte subtypes and for proper antibody isotype control and fluorescence compensations to reduce the background effect to allow for interpretation of true biomarker dose/time-kinetics after exposure to X-rays (FIG. 11).

Human ex-vivo blood irradiations are performed using an X-Rad 320 Irradiator (Precision X-Ray). The irradiator is equipped with a custom-made Thoraeus filter (1.25 mm Sn, 0.25 mm Cu, 1.5 mm Al). Dose rate from the X-Rad 320 is calibrated periodically using a factory-calibrated Accudose 10×6-6 Ionization Chamber. The dose rate is 1 Gy/min.

VFI tolerates most of detergents in assay buffer. We will favor Triton X-100 as detergent for the cell lysis buffer as it is already present in the VFI assay buffer but if lysis is not efficient enough, buffer composition will be adapted/complemented with other reagents (e.g., NP-40, SDS, etc.).

Development of protein sandwich immunoassay and integration in the VFI platform:

The VFI platform can perform the multiplex detection of several proteins, including C-reactive protein (CRP) and total immunoglobin M on the same membrane, from blood, in less than 20 minutes (FIG. 9A). In addition, the VFI has also shown its ability to predict absorbed radiation dose by quantifying simultaneously two CDKN1A and DDB2 biodosimetry genes and providing dose-response curves with the same performance level as the gold standard qRT-PCR (FIG. 9B). The objective is to optimize matched antibodies pair for the sandwich immunodetection of DDB2, BAX, phosphor-p53 (Ser 37), FDXR, ACTNI and TSPYL2 radiation dosimetry proteins, along with ß-tubulin as a housekeeper protein for normalization, identified for their integration in the VFI device. In this manner, any number and type of sandwich immunodetection capture agents can be incorporated into a VFB of the device.

Commercially available antibodies for the FAST-DOSE biomarkers and protein

standard are used to develop the sandwich immunoassay. The capture/detection antibodies pair with the highest sensitivity for each protein are first empirically determined after testing all possible combinations using standard approach on microwell plate. Once determined, the two best antibody pair are tested on the VFI. To start, only one protein per membrane is processed. First, different printing protocols are tested in order to determine the optimal concentration of the coated detection antibody on the VFI membrane. Then, 5 Gy-irradiated samples are used as positive control, and will be run to assess efficiency of the detection protocol. The VFI uses gold nanoparticles (AuNP) conjugated antibodies to provide a signal visible with naked eyes and/or standard smartphone camera. For the detection, two strategies can be used to optimize this signal with either a direct conjugation of the detection antibody with the AuNP or an AuNP-conjugated anti-FITC antibody to bind a FITC-conjugated detection antibody. After single detection, multiplex membranes are precisely printed using a GESiM nanoplotter, and irradiated samples are run individually (i.e., with the presence of detection antibodies for one protein) to assess the assay specificity and optimize it if needed. Finally, a full assay is run to assess the simultaneous detection of the proteins. Non-irradiated samples will also be included to evaluate the VFI ability to detect expression level changes between sham-and irradiated samples.

Preliminary results show that the VFI can multiplex protein detection and provide dose-dependent response of radiation dosimetry biomarkers. Although VFI have shown superiority and is best adapted for multiplexing compared to others POC assays, the main complexity of this assay will reside in the detection of 6 different proteins+normalizer. If specificity is not satisfied for 6 proteins, then selection will be decreased and optimized to allow detection of 3 proteins for the immediate purpose of this project. In case of a challenging development and integration of the β-tubulin housekeeper protein, additional candidates (e.g. SDHA, TFRC or β2M) can be tested as an alternative.

Comparison of VFI and Imaging Flow Cytometry techniques for detection of FAST-DOSE biomarkers: One major step for the validation of new POC-VFI clinical devices is to ensure a sensitivity and accuracy at least equivalent to the reference method while providing a convenient and simple interface for the final user. The VFI has already gone through user interface evaluation for its experimental demonstration during the Inspiration4 space mission and during a demonstration for multiplex biothreats detection. Thus, the VFI has been tested by untrained and non-specialist in extreme conditions (i.e., microgravity; mobile field operation) where its rapid and easy workflow allowed the users to run an entire assay.

From the same non-irradiated and irradiated blood samples, we prepare sample aliquots (100 μl) in 96-well format for automated capture and cellular image acquisition and quantification using the ImageStreamX® IFC and IDEAS® software, respectively [1,10]. For each protein biomarker (FDXR, ACTNI, DDB2, BAX, phospho-p53 (p53), and TSPYL2, see FIG. 11), mean fluorescence intensity (MFI) in the entire population is measured. Dose response curves based on fold change is constructed for each biomarker.

In this manner, the optimal FAST-DOSE markers signature are used on the VFI to provide a reliable tool to be used directly on-site and to rapidly screen individuals exposed to a dose <2Gy from a dose ≥2 Gy. Key factors include both the sensitivity of the VFI and its ability to be used anywhere by non-trained personnel. In regard to VFI performance, we do not anticipate any problems as the VFI already demonstrated a high sensitivity, 10 times better than the ELISA [4]. In addition, as previously mentioned, the VFI has also been used in remote environment. During these tests, our data show that the VFI device can be easily shipped (FIG. 12), and the reagents are stable for more than a month at room temperature with appropriate packaging. Second, these tests demonstrated that inexperienced users could perform the test by themselves.

### Example 8: A Self-Sustained Non-Invasive and Multiplex Self-Diagnostics Platform for Detecting SARS-COV-2 Virus and Neutralizing Antibodies in Biofluids

Provided herein are devices for multiplexing of molecular assays into a self-sustained platform which does not require any electrical power to have a rapid and safe analysis run, by any individual or consumer, at any point of care or at home, from a self-collected sample of saliva, oral swab, fingerstick blood droplet or other biofluids. Results can be visualized directly or imaged by a smartphone appliance which can be equipped with VeriFAST application software. The proposed kit can run, using the same platform, SARS-CoV-2 neutralizing antibodies tests in less than 15 minutes and gene expression assay for the SARS-COV-2 virus detection in about 40 minutes, depending on the required limit of detection and type of biomarker(s). The multiplexing of detection devices described herein are suitable for other panels of biomarkers of other respiratory viruses or emerging pathogens. The safe ease-of-use, at home, self-sustainable and low cost configuration of this “direct-to-consumer kit” using self-collection of saliva or blood, makes it readily scalable for mass production.

The device (FIG. 12 (illustrating OMTEST device)) is novel and significantly advanced compared to other work in the field. It can detect genes and proteins, including without any battery-powered reader. We have demonstrated its versatility in applications and operations ranging from detecting Tier-1 biothreats, SARS-COV-2 genes, antigens and antibodies in human saliva and blood up to performing gene-based biodosimetry and monitoring health performance of astronauts in zero-gravity environment (Space X Inspiration 4 flight launched on Sep. 15, 2021). The safe and simple operation of the device is compatible for truly home-based or mobile self-test by individuals in urban or low resources settings across the world.

The device and methods provided herein address an important public health threat and can be useful to help facilitate providing medical countermeasures, especially under a pandemic emergency. A rapid diagnostic modality that can be self-sustained and self-administered frequently for “all-population” following emerging infectious diseases exposure is relevant scientifically, economically and as an overall security priority. The devices and methods are a first comprehensive user-centered medical countermeasure that can be used in a pandemic or after other chemical, biological, radiological and/or nuclear (CBRN) event. The devices provided herein provide the capability to monitor more frequently, including ranging from local to globally exposed populations with a simple, autonomous and affordable self-test.

Point-of-care (POC) assays are essential diagnostic platforms that allow fast health monitoring and disease testing with minimal equipment. Their use has shown to improve both clinical and economic outcome by, for example, allowing faster decision, starting treatment earlier, reducing use of staff, equipment, and hospital admissions. In a context of pandemic such as the COVID-19, their use would drastically improve public health management and outcome.

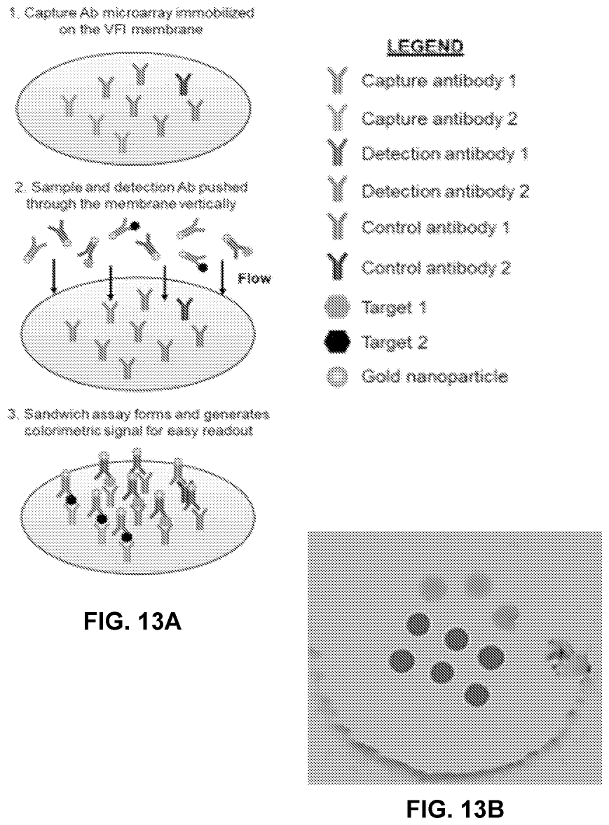

As an example, provided is a multiplexed detection of eight Tier 1 biothreat agents using a VFB that is a Vertical Flow paper-based Immunoassay (VFI). Using a miniaturized “syringe-like” device, the sample is pushed through a membrane, which is pre-functionalized with capture antibody, a sandwich assay is then formed and a colorimetric signal is generated by gold nanoparticle (AuNP) to reflect the presence of target antigens (FIGS. 13A-13B). Thus, we have demonstrated the functionality and advantage of the VFI platform with an improved limit of detection >80+ vs. standard lateral flow immunoassay (LFI) for the detection of the bio-threat pathogen Burkholderia pseudomallei, for example. These results show the ability of VFI to detect microbial antigens or more generally protein markers.

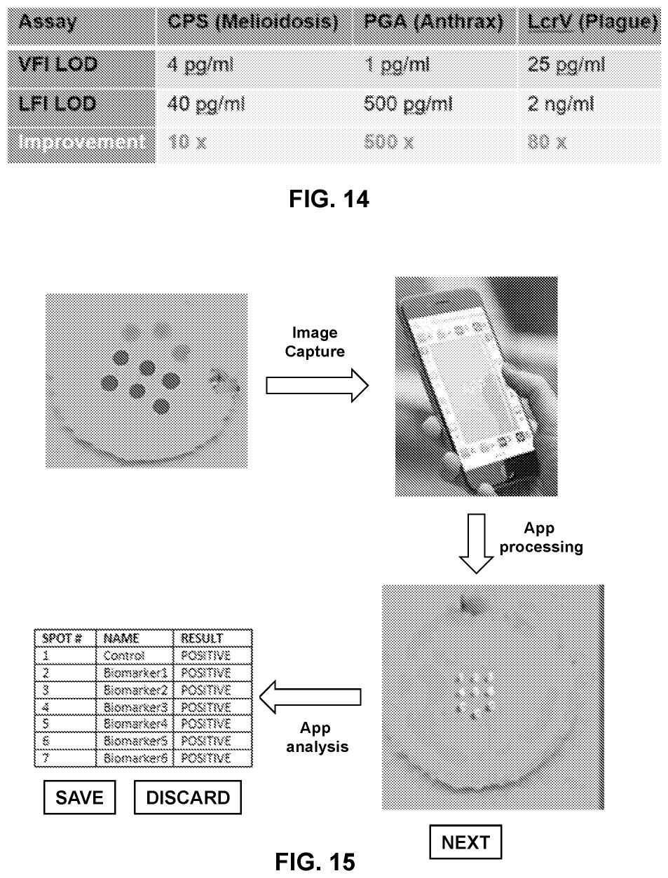

The main advantage of the VFI system over the traditional LFI platform is that it allows a multiplexed detection of large amounts of biomarkers simultaneously. Plus, the flow path of the VFI is only through the thickness of the porous membrane (˜130 um) vs. ˜40 mm length in the LFI. This short flow path allows nanoporous membranes to be used for more efficient target capture, as well as larger volumes of sample to be processed than LFI. A miniaturized Silicon support may be used to reduce the signal variation across the membrane for better multiplexed biomarkers detection and improved sensitivity. For example, FIG. 14 a table of some of the multiplex assays revealing large improvement in limit of detection (LOD) of Tier1 biothreats.

Further advances are obtained using novel gold stars nanoparticles and device miniaturization. According to scaling laws of the system, reducing the diameter of the membrane from 10 mm to 2.5 mm, the sample and reagent consumption is reduced by 16×; the active pumping pressure can also be increased 5× to pushing more sample through the membrane; and higher concentration of AuNP can be used for better assay sensitivity. Based on this miniaturized design, a POC device, or Vertical Integrated Flow Assay System Technology (VeriFAST), is developed for mobile missions (FIG. 15). The kit includes an automated syringe pump, which can maintain constant flow rate during the assay.

Vertical Flow Assay for Multiplex Gene Expression: The VeriFAST technology is compatible with additional functionality to facilitate the detection of nucleic acids. Indeed, by using the same immunodetection approach, we developed an assay to amplify and quantify RNA levels of two biodosimetry and one housekeeping genes from blood cells in order to monitor absorbed radiation dose in mobile and remote environment, such as during deep space flights. The bioassay showed that it can detect the expression level of two biodosimetry genes (CDKN1A and DDB2) while using a housekeeping gene (MRPS5) for normalization with a high specificity (FIG. 8). The data also demonstrates that, similar to qPCR, the VFI platform detects an increase in expression level of these biodosimetry genes in function of the dose (FIG. 9B), suggesting that the VFI can be used to monitor absorbed radiation dose and, at large, to measure efficiently expression of gene biomarkers.

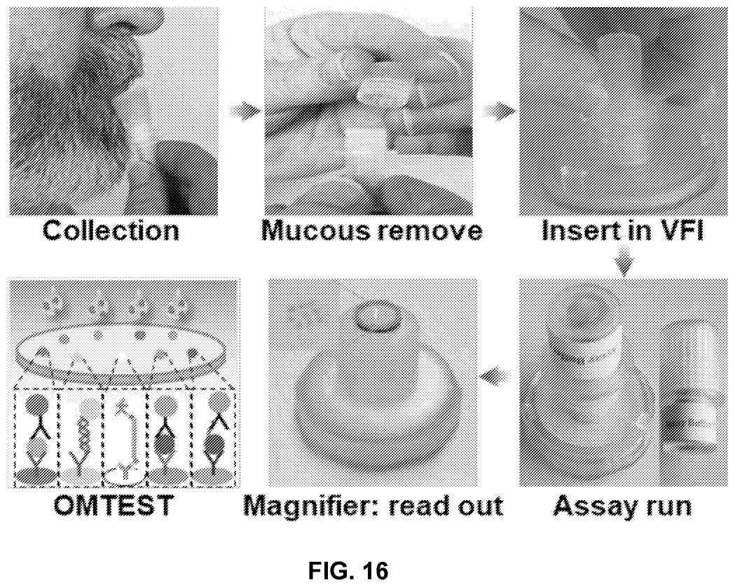

Vertical Flow Assay for Multiplex COVID-19 Diagnostics: With the sudden COVID-19 pandemic, there is a need to develop rapid sample-to-answer POC devices suitable for ease-of-use by individuals everywhere. It is also critical to accommodate multiple assay chemistries including the PCR-based gene detection of the viral infection, but also antibodies indicative of the immunity level of an individual. For this purpose, we designed a device to allow a user to self-collect saliva and process the biospecimen directly into the VFI device without the need of any electrical or battery powered instrumentation, and that can be performed at home, in less than a few minutes (proteins) to about half-hour (genes), from sample collection to data analysis. The device is based on a simple system of in-and-out plugged caps where assay buffers and reagents are contained in absorbing pads sealed in caps, thus reducing the risk of exposure to hazardous chemicals, and does not require any electronic instrumentation (e.g. syringe pump or thermocycler) since fluid is moved by capillarity. The different parts comprise an integrated saliva collector, a pad holder containing the stack of filters, pads and membranes, assay and washing buffer caps and a magnifier for a direct visualization by naked-eye or using the VeriFAST APP on a smartphone (iPhone). This process (FIG. 16) is safe and simple for everybody everywhere to use.

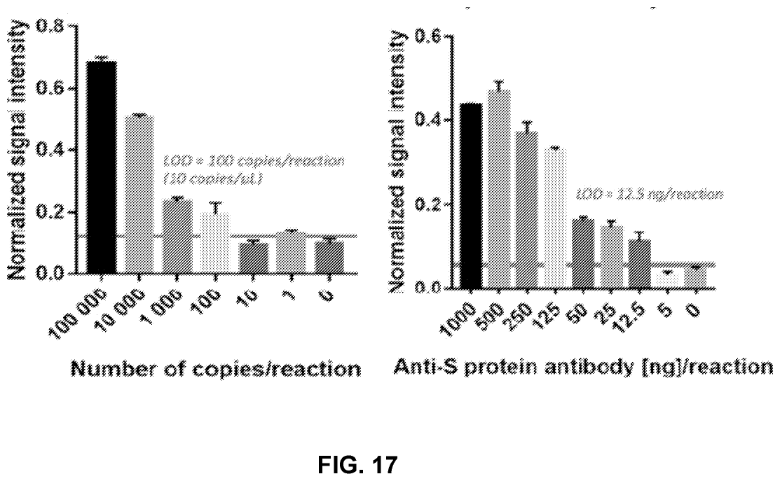

Using this device, we successfully demonstrate the capacity to detect in saliva both gene (SARS-COV-2 N gene to detect viral particles) and protein (human anti-S protein IgG to assess immune response to SARS-COV-2). We established preliminary limit-of-detection of 10 copies/μL for SARS-COV-2 viral particles and about 12.5 ng/reaction for anti-S SARS-COV-2 protein IgG (FIG. 17). These results fit within the specifications of the different commercial products using single analytes and which received FDA EUA.

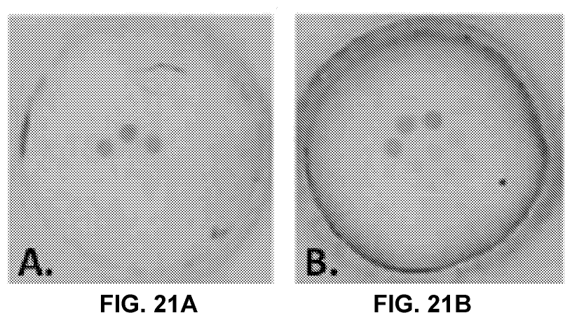

More interestingly, we also demonstrate that both N gene and anti-S protein IgG spiked in saliva fluid could be identified on the same membrane (FIGS. 10A-10B), showing the capacity of the system for multiplex detection of both gene and proteins, and so, in the context of COVID-19, detecting both viral load and humoral response. Finally, we also showed that the device is compatible with different matrixes by also detecting anti-S protein IgG in plasma. Under IRB study protocol, plasma from volunteers who received Pfizer and Moderna vaccines, or recovered from COVID-19 were collected from fingerstick and showed positive results while control plasma collected before the start of the pandemic shows a negative signal (compare FIG. 21A and 21B).

### Validation in Various POC Settings

In order to validate this technology as a robust POC bioassay, the device is tested in extreme conditions during the all-civilian crew flight Inspiration 4 for a four day mission onboard of Space X Dragon capsule in orbit. For this purpose, the detection of one inflammatory biomarker (C-Reactive protein (CRP)) and one immune biomarker (IgM), both secreted in plasma, has been achieved (FIG. 6B). Typically, CRP high concentration in plasma (less than 3 μg/mL for normal conditions) hampers its detection and quantification in POC devices set up by the Hook effect. Using the device, we were able to detect CRP directly from plasma (2-10 μL) without any dilution or sample preparation (FIG. 5A). A wide range of concentrations (1-20 μg/ml) of CRP was detectable in VFI setup after only 2.5 min, with the strongest signal being obtained at 15 min (FIG. 5B). Importantly, this setup allows us to evaluate stability of the OMTEST, and an ongoing study shows detection antibodies immobilized on the conjugation pad stay functional for more than 1 week (FIG. 5C). For Inspiration 4, these conditions have been successfully extended to more than 8 weeks. In addition, this work highlighted important features of the device for POC application, and particularly the simplicity of user interface (i.e. use by untrained, non-scientists users) and robustness of operations in extreme environments (e.g. microgravity).

Platform Optimization: We have demonstrated that the device can detect antibodies against SARS-COV-2 Spike glycoprotein in human blood and saliva. For this latter biofluid, we specifically developed a polymeric-based solution that can interact with mucin to decrease saliva viscosity, not only by binding mucin, but also by trapping debris and food residues. By integrating this step in the device and process, and as a proof of concept, the device was able to detect, in less than 15 min, up to 85 ng of recombinant anti-SARS-COV-2 S-protein IgG spiked in real saliva.

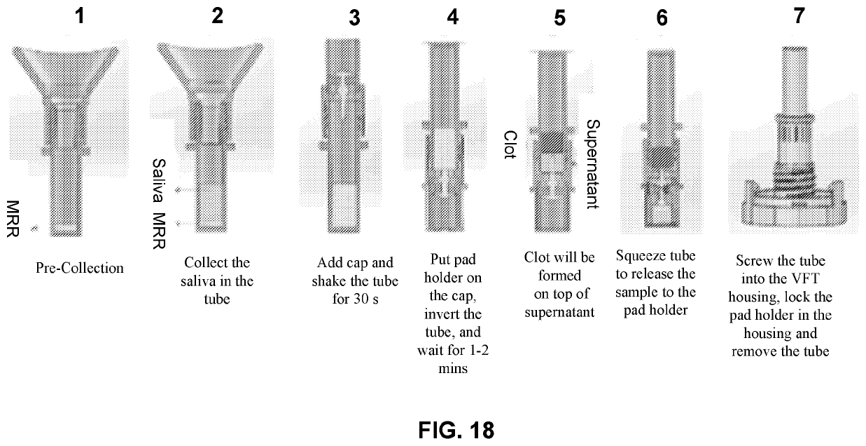

Design optimization of saliva collector and device integration: Saliva collection and pre-processing with polymer-based mucus removal reagents is automated and compatible with the device cap/tube configuration (FIG. 18). Design and 3D rapid prototyping is optimized (e.g. valve/metering).

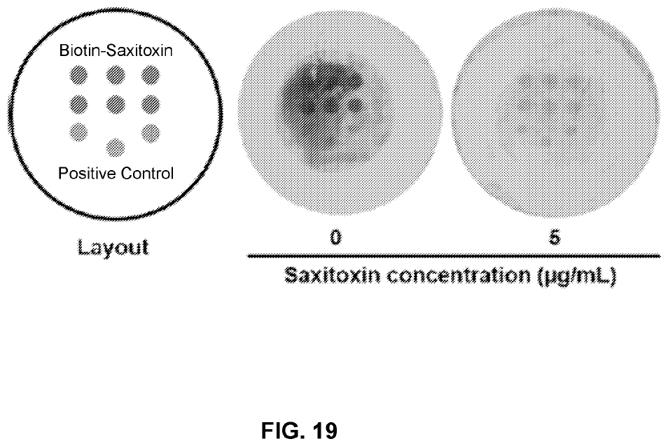

Device to detect of SARS-COV-2 neutralizing human antibodies: We showed that the platform technology was able to detect binding human antibodies against SARS-CoV-2 Spike glycoprotein using a sandwich immunoassay approach in plasma and saliva samples. We adapt and translate this approach into a competitive immunoassay to specifically detect neutralizing antibodies by their ability to inhibit the binding with ACE2 receptor and protein S RBD domain. Interestingly, we also demonstrate the capacity of the VFI platform to be used as a competitive assay to detect small molecules. In this assay, a biotin-conjugated saxitoxin is coated on the VFI membrane. Sample is then incubated with a gold-nanoparticle conjugated anti-saxitoxin antibody for 10 min before being passed through the membrane. Results reflect, as expected, in presence of saxitoxin in the sample the signal decreases while the saxitoxin-free sample displays a strong signal because of the non-competitive binding with the anti-saxitoxin antibody (FIG. 19).

Therefore, for this development stage, we develop such competitive assay for the detection of SARS-COV-2 neutralizing antibodies. Both combinations, including capture ACE2 receptor with gold-nanoparticle conjugated recombinant S-protein or capture SARS-CoV-2 Spike glycoprotein with gold-nanoparticle conjugated ACE2 receptor will be first tested. The different configurations are then assessed by spiking a S-protein neutralizing antibody at different concentrations incubated with either gold nanoparticle conjugated ACE2 receptor or SARS-COV-2 Spike S1 RBD glycoprotein. Signal intensity is quantified, and dose-response curves are assessed as the % inhibition (decrease of signal intensity) per antibody concentration. Protocol optimization focuses on decreasing the assay time, increasing sensitivity and minimizing background and noise-to-signal ratio by testing different buffer, incubation time and membrane material.

Detection of SARS-COV-2 N gene and control using isothermal amplification: Using a benchtop sample lysis and purification protocols, our preliminary data showed that the VFI-membrane could be used to detect at least 100 copies of viral RNA/reaction (10 copies/μL) after isothermal amplification (FIG. 17). In order to perform gene detection on the device, the protocol for the three major steps (i.e. sample preparation, amplification, purification) is required to be adapted (e.g. magnetic beads or filtration) to avoid powered large instruments and harsh treatments (e.g. heat, sonication, or high centrifugation).

“Sustained/powerless” module for isothermal amplification: We have developed a one-step isothermal amplification benchtop protocol that can use a constant and low temperature (˜42° C.) to perform both reverse transcription and amplification. Therefore, we also have established a low-cost, user-friendly, and “power-less” isolation box to provide a tunable temperature range of 38-42° C. using air-activated disposable hand warmer products that are already available in the market and approved in terms of user safety (FIG. 2A-2D). After thermal stabilization in the kit carton box, our data showed that an amplification for about 20 min using this setup was as efficient as benchtop protocol to detect 100 copies of viral RNA. The current configuration will require optimization of the heat-actuation and materials to reach the desired temperature more rapidly and facilitate reproducible user interface.

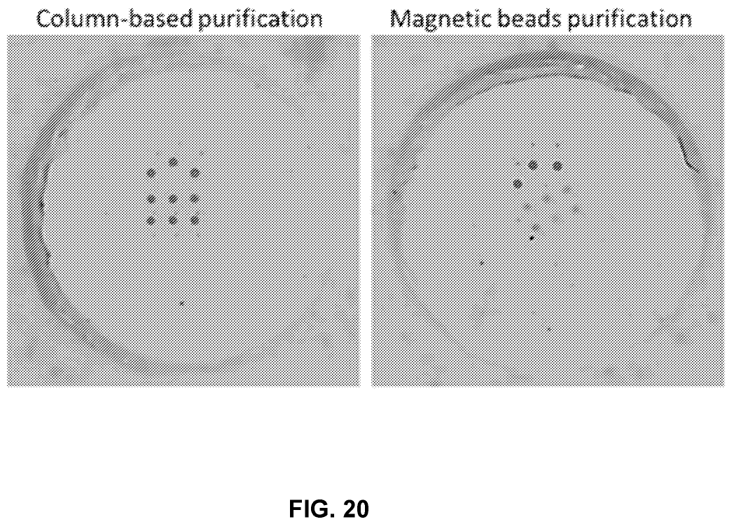

Purification protocol: The simple nucleic assay included in the OMTEST requires a purification step at the end of the amplification in order to both reduce background and improve sensitivity. A current protocol employs column-based purification using high centrifugation speed. However, we already showed that this protocol can be replaced by a magnetic bead approach that could be integrated in a POC “powerless” device (FIG. 20).

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

1. Peng Chen, Marcellene Gates-Hollingsworth, Sujata Pandit, Anson Park, Douglas Montgomery, David AuCoin, Jian Gu and Frederic Zenhausern, Paper-based Vertical Flow Immunoassay (VFI) for detection of bio-threat pathogens, Talanta, 2019, 191:81-88. https://doi.org/10.1016/j.talanta.2018.08.043.
2. Jasmine Pramila Devadhasan, Jian Gu, Peng Chen, Stanley Smith, Baiju Thomas, Marcellene Gates-Hollingsworth, Derrick Hau, Sujata Pandit, David AuCoin and Frederic Zenhausern, Critical Comparison Between Large and Mini Vertical Flow Immunoassay Platforms for Yersinia Pestis Detection, Analytical Chemistry, 2021, 93, 27, 9337-9344 (incl. front cover). doi: 10.1021/acs.analchem.0c05278.
3. Ngan N. T. Nguyen, Colleen Mccarthy, Darlin Lantigua and Gulden Camci-Unal, Development of Diagnostic Tests for Detection of SARS-COV-2. Diagnostics (Basel). 2020 Nov. 5; 10 (11): 905. doi: 10.3390/diagnostics10110905. PMID: 33167445; PMCID: PMC7694548.
4. Wang Q, Lee Y, Shuryak I, Pujol Canadell M, Taveras M, Perrier JR, et al. Development of the FAST-DOSE assay system for high-throughput biodosimetry and radiation triage. Sci Rep. 2020; 10:12716. doi: 10.1038/s41598-020-69460-7
5. Taraboletti A, Goudarzi M, Kabir A, Moon B-H, Laiakis EC, Lacombe J, et al. Fabric Phase Sorptive Extraction-A Metabolomic Preprocessing Approach for Ionizing Radiation Exposure Assessment. J Proteome Res. 2019; 18:3020-3031. doi: 10.1021/acs.jproteome.9b00142
6. Lee Y, Wang Q, Shuryak I, Brenner DJ, Turner HC. Development of a high-throughput γ-H2AX assay based on imaging flow cytometry. Radiat Oncol. 2019; 14:150. doi: 10.1186/s13014-019-1344-7
7. Garty G, Xu Y, Johnson GW, Smilenov LB, Joseph SK, Pujol-Canadell M, et al. VADER: a variable dose-rate external 137Cs irradiator for internal emitter and low dose rate studies. Sci Rep. 2020; 10:19899. doi: 10.1038/s41598-020-76941-2
8. Shuryak I, Turner HC, Perrier JR, Cunha L, Canadell MP, Durrani MH, et al. A High Throughput Approach to Reconstruct Partial-Body and Neutron Radiation Exposures on an Individual Basis. Sci Rep. 2020;10:2899. doi: 10.1038/s41598-020-59695-9
9. Wang Q, Rodrigues MA, Repin M, Pampou S, Beaton-Green LA, Perrier J, et al. Automated Triage Radiation Biodosimetry: Integrating Imaging Flow Cytometry with High-Throughput Robotics to Perform the Cytokinesis-Block Micronucleus Assay. Radiat Res. 2019;191:342-351. doi: 10.1667/RR15243.1
10. Wang Q, Pujol-Canadell M, Taveras M, Garty G, Perrier J, Bueno-Beti C, et al. DNA damage response in peripheral mouse blood leukocytes in vivo after variable, low-dose rate exposure. Radiat Environ Biophys. 2020;59:89-98. doi: 10.1007/s00411-019-00825-x
11. Lee Y, Pujol Canadell M, Shuryak I, Perrier JR, Taveras M, Patel P, et al. Candidate protein markers for radiation biodosimetry in the hematopoietically humanized mouse model. Sci Rep. 2018;8:13557. doi: 10.1038/s41598-018-31740-8.
12. Williams BB, Flood AB, Demidenko E, Swartz HM. ROC Analysis for Evaluation of Radiation Biodosimetry Technologies. Radiat Prot Dosimetry. 2016;172:145-151. doi: 10.1093/rpd/ncw168
U.S. Pat. Pub. 2021/0199651 titled “Vertical Flow Molecular Assay Apparatus”
PCT Pub. WO2022/072876 titled “Biofluid Self-Collector and Processing Device”

1. A device for self-testing for a plurality of biomarkers comprising:
a sample inlet configured to receive a liquid biofluid sample;
a sample pre-processing module fluidically connected to the sample inlet to provide a pre-processed biofluid sample;
a filter in fluidic contact with the liquid biofluid sample or the pre-processing module to provide a filtered fluid sample;
a powerless heat source in thermal contact with the pre-processed biofluid sample and/or the filtered fluid sample for controlled temperature of the pre-processed biofluid sample and/or filtered fluid sample;
a vertical flow biosensor (VFB) comprising a multiplex membrane in fluidic contact with the pre-processed biofluid sample for multiplex detection of the plurality biomarkers in the liquid biofluid sample.

2. The device of claim 1 that is self-powered without any external electrical power source.

3. The device of claim 1, wherein the liquid biofluid sample is a saliva sample, a plasma sample, a blood sample, a urine sample, a sputum sample, a semen sample, a vaginal discharge sample, a tear fluid, a breath condensation droplet, a CSF fluid biopsy or plural effusion.

4. The device of claim 1, wherein the sample pre-processing module comprises one or more of:
the filter in fluidic contact with the liquid biofluid sample to provide the pre-processed biofluid sample that is the filtered biofluid sample;
a mucus removal reagent (MRR) fluidically connected to the sample inlet to introduce the MRR to the liquid biofluid sample that is a saliva sample, wherein MRR removes mucus from the saliva sample and reduces a viscosity of the saliva sample;
a rheological property adjuster;
a pH adjuster;
a concentration adjuster; or
an interaction force modulator.

5. The device of claim 1, wherein the plurality of biomarkers comprises one or more of:
one or more markers of an infectious agent and fragments thereof, including polypeptides and/or polynucleotides;
one or more markers of a host immune response;
one or more vaccine markers;
one or more cancer biomarkers;
one or more nutrition or metabolic biomarkers;
one or more auto-immune disorder biomarkers;
one or more cardiovascular biomarkers;
one or more genetic disorder biomarkers; or one or more environmental biomarkers.

6. The device of claim 1, wherein the powerless heat source comprises: a chemical heat source comprising reagents for an exothermic chemical reaction to provide a biological sample temperature range of between 34° C. and 95° C. to activate at least one step of an amplification reaction of a biological component in the liquid biofluid sample.

7. The device of claim 6, wherein the amplification reaction is by PCR; LCR; isothermal; and/or RCA.

8. The device of claim 4, wherein the MRR comprises a polymeric-based solution configured to interact with mucin in the liquid biological sample that comprises saliva, the device further comprising:
a filter substrate material having an average pore size selected to remove debris and food residue from the saliva; and
a substrate material having a physical parameter and chemical property configured to establish an interface with the biofluid sample for directing one or more analytes in the biofluid sample to the VFB.

9. The device of claim 1, wherein the VFB is an electromagnetic power-free VFB that is fluidically activated by intra-molecular or external forces, comprising:
a membrane housing; and
a sample absorbent pad in fluidic contact with the membrane, wherein the multiplex membrane and the sample absorbent pad are positioned in the membrane housing and the absorbent pad is fluidically connected to the liquid biological sample, wherein biomarkers from the biological liquid sample are provided to the multiplex membrane.

10. The device of claim 1, wherein the VFB is in a stacked-pad configuration comprising:
a buffer pad configured to store assay buffer;
a sample absorbent pad;
the multiplex membrane, such as a polyethersulfone membrane;
a conjugation pad;
a retarding pad; and
a flow directing pad.

11. The device of claim 1, further comprising an imager for optical detection of presence or absence of the plurality of biomarkers in the VFB, wherein the imager is optionally a magnifying lens and/or a portable reader.

12. The device of claim 11, wherein the imager comprises a magnifying lens configured to optically align with at least one lens of a smart phone or a commercially-available ancillary configured to perform biomarker analysis.

13. The device of claim 1, further comprising a point-of-care microfluidic cartridge for preparing proteins in the liquid biological sample for detection by the VFB.

14. The device of claim 1, wherein the biomarkers comprise one or more of:
human antibodies to an infectious agent, such as human anti-SARS-COV2 antibodies;
total IgM, IgC, IgA, or combinations thereof;
inflammatory or stress response protein(s), such as CRP;
SARS-COV-2 N-gene;
Human CDKN1A, DDB2 and MRPS5 gene; and/or
a small molecule or other biomolecular species indicative of a disease condition or an environmental exposure.

15. The device of claim 1, wherein biomarker detection is independent of device orientation and operable under zero-g conditions.

16. The device of claim 1, wherein the multiplex membrane comprises biomarker detectors to detect:
presence or absence of a virus; and
presence or absence of a host immune response.

17. A method of detecting a biological parameter, the method comprising the steps of:
providing the device of claim 1;
introducing the liquid biofluid sample to the sample inlet;
removing debris in the liquid biological sample by the pre-processing module;
introducing the pre-processed filtered liquid biofluid sample mixture to the multiplex membrane; and
optically detecting the one or more biomarkers that have interacted with the multiplex membrane,
thereby detecting the biological parameter.

18. The method of claim 17, wherein the removing step comprises: mixing the liquid biofluid sample with a MRR and filtering the mixed liquid biofluid sample and MRR to provide a filtered liquid biological sample.

19. The method of claim 17, having a total method time of less than one hour.

20. The method of claim 17, wherein the biological parameter is one or more of:
determination of a past infection event;
current infection status;
immunity status;
donor compatibility;
vaccine quality control;
radiation biodosimetry;
prediction of treatment efficacy;
risk assessment of disease susceptibility;
screening/detection: indication of the presence of the disease;
assessment of disease aggressiveness for prognosis;
monitoring of disease recurrence and therapeutic response; and/or
pharmacological response, including drug efficacy, dose response, safety or genotype.
